(19) United States
(12) Patent Application Publication
(10) Pub. No.: US 2007/0065860 A1
(43) Pub. Date: Mar. 22, 2007

(54) Accelerated class I and class II HLA DNA sequence-based typing

(76) Inventors: William H. Hildebrand, Edmond, OK (US); Steven Cate, Norman, OK (US); Runying Tian, Norman, OK (US)

Correspondence Address: DUNLAP, CODDING & ROGERS P.C., PO BOX 16370, OKLAHOMA CITY, OK, 73113, US

(21) Appl. No.: 11/523,981

(22) Filed: Sep. 20, 2006

Related U.S. Application Data
(60) Provisional application No. 60/718,651, filed on Sep. 20, 2005.
(60) Provisional application No. 60/731,084, filed on Oct. 28, 2005.

(51) Int. Cl. C12Q 1/68; C12P 19/34

(52) U.S. Cl. 435/6; 435/91.2

(57) ABSTRACT

There is provided a method for directly typing or sequencing HLA class I or class II alleles from a tissue sample wherein at least one exon of the HLA class I or class II alleles from the sample is amplified in a locus specific manner utilizing two primers, wherein at least one of the primers has a universal or generic sequencing primer site incorporated therein. After amplification, the amplified exon(s) are directly sequenced, and a comparison is made between the derived HLA allele sequence and an HLA allele database, thereby giving an exact HLA type for the sample being tested.

### CROSS REFERENCE TO RELATED APPLICATIONS

This application claims benefit under 35 U.S.C. 119(e) of provisional applications U.S. Ser. No. 60/718,651, filed Sep. 20, 2005; and U.S. Ser. No. 60/731,084, filed Oct. 28, 2005. The contents of each of the above-referenced patent applications are hereby expressly incorporated herein by reference in their entirety.

### FEDERALLY SPONSORED RESEARCH OR DEVELOPMENT

Not Applicable.

### BACKGROUND OF THE INVENTION

1. Field of the Invention

The present invention relates in general to a method for direct DNA sequence based typing of HLA class I and class II alleles, and more particularly, but not by way of limitation, the present invention is directed to the sequence based typing of at least one exon of the HLA allele gene under study.

2. Brief Description of the Related Art

MHC has the highest genetic polymorphism of the mammalian DNA molecules. Questions are raised by this polymorphism, such as its molecular basis, degree, and functional significance. For analysis of MHC polymorphism and its relationship to immune responses and disease susceptibilities, the human species has considerable scientific advantages, as well as direct relevance to clinical medicine. Only in human populations is there likely to be extensive analysis of MHC polymorphism from many geographically separated populations. The crystal structure of the human class I molecule has also been previously disclosed, making accurate insights into other HLA class I and class II molecules possible as well as the allelic polymorphism of the HLA-A, B, C, DR, DP and DQ genes.

The HLA class I and class II genes are a component of the human major histocompatibility complex (MHC). The class I genes consist of the three classical genes encoding the major transplantation antigens HLA-A, HLA-B and HLA-Cw and seven non-classical class I genes, HLA-E, HLA-F, HLA-G, HLA-H, HLA-J, HLA-K, and HLA-L. The class II genes are encoded in the class II region of the human HLA complex at the DR, DQ and DP loci.

The classical HLA class I and class II genes encode polymorphic cell surface proteins. Class I molecules are expressed on most nucleated cells, while Class II molecules are expressed on a limited number of cells, including but not limited to, dendritic cells (DC), B cells, macrophages, and the like. The natural function of these Class I and Class II proteins is to bind and present diverse sets of peptide fragments from intracellularly processed antigens (Class I) and extracellular antigens (Class II) to the T cell antigen receptors (TCRs). Thus, the peptide-binding capacity of the MHC molecule facilitates immune recognition of intracellular pathogens and altered self proteins (Class I) and extracellular pathogens and allergens (Class II). Therefore, by increasing the peptide repertoire for TCRs, the polymorphism of MHC molecules plays a critical role in the immune response potential of a host. On the other hand, MHC polymorphism exerts an immunological burden on the host transplanted with allogeneic tissues. As a result, mismatches in HLA class I and/or class II molecules are one of the main causes of allograft rejection and graft versus host disease, and the level of HLA matching between tissue donor and recipient is a major factor in the success of allogeneic tissue and marrow transplants. It is therefore a matter of considerable medical significance to be able to determine the “type” of the HLA class I and/or class II genes of candidate organ donors and recipients.

HLA class I and class II histocompatibility antigens for patient-donor matching are conventionally determined by serological typing. Biochemical and molecular techniques have revealed that HLA class I and class II polymorphism are far greater than previously recognized by conventional methods. To date, over 472 HLA-A, 805 HLA-B, 256 HLA-Cw, 541 HLA-DR, 34 HLA-DQA1 and 73 HLA-DQB1 different allelic sequences have been identified. This high level of allelic diversity complicates the typing of the HLA class I and class II genes.

Another complicating factor is the large number of class I and class II alleles that are heterogenous in their DNA and amino acid sequence. Each of the HLA class I genes is composed of eight exons and seven introns, and the sequences of these exons and introns are not tightly conserved across the HLA class I genes. Allelic variations occur most frequently in exons 2, 3 and 4 of the class I alpha chain. These two exons are flanked by noncoding introns 1, 2, and 3, and these introns are also polymorphic. These two exons encode the functional domains of the molecules. Variation among class II genes is concentrated in exon 2 of the beta chain and exon 2 of the alpha chain. Intron 1 and intron 2 flanking exon 2 of the HLA class II alpha and beta chains have extensive polymorphism, creating complexity in locating locus-specific PCT primers in conserved intronic regions while eliminating amplification of other loci. This is especially important for HLA-DRB1 separation from HLA-DRB3/4/5.

Taken together, these two complications make HLA class I and class II typing at the nucleic acid level a formidable task. Allelic diversity within any one gene means that a great many DNA probes need to be developed if hybridization-based tests are used in the typing. Further, the general applicability of DNA typing methods to HLA class I and class II genes depends on the design of PCR primers which provide effective locus-specific amplification of exons 2, 3 and/or 4 of one HLA class I gene and exons 2 and/or 3 of HLA class II. Design and application of such locus-specific PCR primers in either introns and/or exons is complicated by extensive polymorphism.

One method for performing HLA class I typing is disclosed in U.S. Pat. No. 5,424,184, issued to Santamaria et al. on May 8, 1991, the contents of which are expressly incorporated herein by reference. This patent utilizes primers which are located within exons 2 and 3 of the HLA class I genes to achieve what is described as group-specific amplification of a portion of the HLA-A, HLA-B, and HLA-C genes. This approach is not ideal, however, since the primers hybridize with portions of the coding strand, and thus may mask significant allelic variations. In addition, this method requires a grouping of alleles by means of another method in order to select group-specific primers for amplification.

Prior to inclusion of polymerase chain reaction (PCR) into the cloning and sequencing of HLA class I and class II alleles, the accumulation of class I sequences was a cumbersome act. The advent of PCR greatly accelerated the accumulation of class I HLA sequences. Since then, research has been undertaken to bring molecularly-based HLA class I and class II typing techniques to the point where they would be clinically useful and cost-beneficial for many applications, such as but not limited to, bone marrow donor registry, disease association, vaccine studies and the like.

Using the bone marrow donor registry as an example, the impetuses driving the development of new semi-automated and automated molecular techniques for high-resolution class I and class II typing could be characterized as: (1) the pool of available donors in the registry were poorly HLA typed: many HLA class I and class II types are of low resolution, and many clinically deleterious class I and class II types were missed; (2) questions pertaining to the discriminatory power of serology had to be addressed: serological HLA typing does not correlate with a high degree of accuracy with the molecular typing of HLA alleles; (3) it is recognized that continual HLA typing will be required to establish and maintain a donor registry of sufficient size; and (4) typing methods based on DNA sequence have many advantages over conventional serologic typing techniques, including the elimination of typing complications due to different tissue distributions of MHC antigens, the use of defined reagents available in unlimited quantities (in contrast to alloantisera which are chemically ill-defined, limited in amount, and frequently monopolized), and the ability to provide an absolute HLA type.

Serologic typing for class II HLA was especially poor because antibodies were able to discriminate few of the many class II HLA. For HLA class II molecules, high resolution clinical typing using the polymerase chain reaction (PCR) with sequence specific primers (SSP) and/or sequence specific oligonucleotide probes (SSOP) became a routine procedure as soon as molecular methods facilitated such typing. This advance in class II typing was essential because alloantisera, traditionally used for class I typing, proved especially inadequate for typing class II antigens. An additional advantage of DNA based HLA typing is that viable cells are not required. While the PCR primers used for amplification of class II molecules in such prior art methods were located in the highly polymorphic exon 2 and therefore resulted in portions of this important exon not being typed, molecular HLA class II typing was still superior to serology. In addition, the understanding of class II polymorphism was immature when the initial class II DNA typing methods were developed; the extent of class II HLA polymorphism was not realized. Thus, the new molecular methods allowed typing of what was known at the time. It has since been learned that there are many more class II alleles, and that the initial molecular methods were not designed to cope with such high levels of class II complexity.

An increasing appreciation of the complexity and the extent of HLA class I and class II polymorphism has continued to develop. The shortcomings of serological typing procedures, and the shortcomings of early DNA based methods, continue to become apparent as more and more alleles are detected. As a logical extension of methods successfully employed for early class II typing, several groups have applied PCR/SSP and/or PCR/SSOP methodologies to molecularly type HLA class I alleles. At first these applications were most successful for the subtyping or low resolution typing of the antigens for which there are multiple variants (such as but not limited to, A2, A*0201/05/06/09/43N/66/75/83N/89/97). These techniques subsequently have required technology improvements to allow for a greater repertoire for the ever growing number of alleles. HLA-A, HLA-B and DRB1 loci provide the most resistance to these typing methodologies.

Accurate class I and class II types must be established for the successful transplantation of organs and bone marrow, for the proper diagnosis of autoimmune disorders such as arthritis, and for research studies trying to establish a link between a particular disease and immune response genes. However, clinical HLA class I and class II typing laboratories (which use antibodies and first generation DNA methods for typing) cannot accurately discriminate among the continuously expanding number of class I and class II genes found in the population. Therefore, more accurate molecular DNA based methods are being sought to shed light on transplantation rejection and autoimmune diseases that continue to escape a solid scientific explanation. Additional precision with class I and class II HLA typing is positioned to complement other scientific advances in our understanding of autoimmune diseases, resistance and susceptibility to infection, resistance and susceptibility to cancer, and transplant outcome.

First generation molecular DNA class I and class II HLA typing methodologies have been unable to cope with extensive polymorphism. Some first generation methods relied on the failure or ability to PCR amplify a gene, with several hundred PCR reactions needed to call a class I or class II type. This is termed SSP (sequence specific PCR). Other techniques utilize one HLA-A, -B, -Cw, -DRB1, -DRB3, -DRB4, -DRB5, -DQA1, or -DQB1 locus specific PCR reaction followed by hybridization with a complex series of oligonucleotide probes. This technique is referred to as SSOP. These techniques utilize a similar first step (an HLA-A, -B, -Cw, -DRB1, -DRB3, -DRB4, -DRB5, -DQA1, or DQB1 locus specific PCR reaction) followed by divergent methods for probing/typing the HLA-A, -B, -Cw, -DRB1, -DRB3, -DRB4, -DRB5, -DQA1, and -DQB1 specific PCR product.

Shortcomings with SSP and SSOP led immunogeneticists to develop DNA “sequence based typing” (SBT) for HLA class I typing. It was posited that SBT would provide a more precise class I HLA type as compared to SSP and SSOP, because SSP and SSOP only give a partial type (i.e., they probe portions of the genes being typed), while SBT reads all of the gene being typed. Although the initial sequence based typing methods were complex and costly, their cost was justified because of the increased accuracy promised by such methods.

One method of SBT is described in U.S. Pat. No. 6,287,764, issued to Hildebrand et al. on Sep. 11, 2001, the contents of which are hereby expressly incorporated herein by reference in their entirety. Such method of SBT describes amplification of HLA-A, -B, and -C class I alleles such that exons 2 and 3 are produced in a locus specific way, then utilizing this PCR product as a template for nested or heminested PCR to produce secondary PCR products in which exons 2 and 3 are amplified separately. These secondary PCR products are produced with an anchoring moiety at one terminus and a DNA sequencing primer at the opposing terminus, such that the secondary PCR products are attached to a solid support for DNA sequencing. However, the methods of the '764 patent do not provide a mechanism by which all of exons 2 and 3 of class I alleles can be sequenced, nor does the '764 patent provide a method of typing class II alleles.

Like the class I SBT strategies, class II typing by DNA sequencing has been plagued by expense and difficulty. In addition, class II DNA SBT has suffered from the fact that PCR primers are located in the extremely polymorphic beginning of exon 2; the placement of PCR primers in exon 2 results in only a portion of exon 2 being HLA typed. Important polymorphisms at the PCR primer sites or immediately adjacent to the PCR primers escape typing and must be typed by another method. This is costly and requires multiple HLA typing platforms. The placement of the primers in introns 1 and 2 flanking exon 2 of the class II loci has an added challenge in developing sequencing primer sites. There are many essential positions near the edges of exon 2 that need to be sequenced, and it is a considerable task to find a conserved position within the intron region for a loci specific sequencing primer.

The ability to attach a universal sequencing site to PCR primers utilized in sequence based typing is greatly hampered by the need to stagger the PCR primers. PCR primers cannot be staggered when heterozygous sequencing is to be initiated from the universal tag attached to the PCR primers, because the resulting sequence would not be decipherable due to overlaps in the sequence.

Therefore, there exists a need in the art for new and improved methods of sequence based typing of HLA class I and class II alleles that overcomes the disadvantages and defects of the prior art. It is to such new and improved methods of sequence based typing that the present invention is directed.

### SUMMARY OF THE INVENTION

The present invention is directed to a method for typing HLA class I or class II alleles. In the method, first and second primers are provided for locus specific PCR amplification of at least one exon of an HLA class I or class II locus. The first primers includes a first portion complementary to the HLA class I or class II locus DNA template, wherein the first portion is located at a 3′ end terminus of the primer, and a second portion not complementary to the HLA class I or class II locus DNA template, wherein the second portion comprises a generic sequencing primer sequence, and wherein the second portion is located at a 5′ end terminus of the first primer. The second primer is complementary to the HLA class I or class II locus DNA template.

A sample comprising at least one HLA class I or class II allele is provided, and at least one exon of the at least one HLA class I or class II allele is amplified in a locus specific fashion utilizing the two primers, thereby providing an amplicon having one generic sequencing primer site at one terminus of the amplicon. The amplicon is then sequenced utilizing a locus-specific sequencing primer and a generic sequencing primer corresponding to the second portion of the first primer to obtain the sequence of the at least one exon of the HLA class I or class II locus.

The HLA allele may be a class I allele or a class II allele. The HLA allele may be selected from the group consisting of HLA-A, HLA-B, HLA-Cw, HLA-DQA1, HLA-DRB1, HLA-DRB3, HLA-DRB4, HLA-DRB5, and HLA-DQB1.

The first portion of the first primer may comprise a nucleotide sequence of at least one of SEQ ID NOS:3-11, 24-27, 35, 45, 46, and 50-70, and the second portion of the first primer may comprise a nucleotide sequence of at least one of SEQ ID NOS:47-49. For example, but not by way of limitation, the first primer may comprise a nucleotide sequence of at least one of SEQ ID NOS:1, 2, 12-23, 28-34 and 36-44. The second primer may comprise a nucleotide sequence of at least one of SEQ ID NOS:3-11, 24-27, 35, 45, 46, and 50-70.

In another embodiment of the method for typing HLA class I or class II alleles of the present invention, first and second primers are provided for locus specific PCR amplification of at least one exon of an HLA class I or class II locus. The first primer comprises a first portion complementary to the HLA DNA template comprising an HLA locus, and a second portion that is not complementary to the HLA DNA template comprising an HLA locus; instead, the second portion of the first primer comprises a forward generic sequencing primer sequence. The first portion of the first primer is located at a 3′ end terminus of the primer, and the second portion of the first primer is located at a 5′ end terminus of the primer. The second primer includes a first portion complementary to the HLA DNA template comprising an HLA locus, and a second portion that is not complementary to the HLA DNA template comprising the HLA locus. The second portion of the second primer includes a reverse generic sequencing primer sequence different from the second portion of the first primer. The first portion is located at a 3′ end terminus of the primer, and the second portion is located at a 5′ end terminus of the primer. The first portions of at least one of the first and second primers may comprise a nucleotide sequence of at least one of SEQ ID NOS:3-11, 24-27, 35, 45, 46, and 50-70, while the second portion of at least one of the first and second primers may comprise a nucleotide sequence of at least one of SEQ ID NOS:47-49. For example but not by way of limitation, at least one of the first and second primers may comprise a nucleotide sequence of at least one of SEQ ID NOS:1, 2, 12-23, 28-23 and 36-44.

A sample comprising at least one HLA allele is provided, and the at least one exon of the at least one HLA allele is amplified in a locus specific fashion utilizing the two primers, thereby providing an amplicon having two generic sequencing primer sites at the opposing terminus of the amplicon such that the generic sequencing primer sites flank the at least one exon to be sequenced. The amplicon is then sequenced utilizing generic sequencing primers corresponding to the second portions of the first and second primers to obtain the sequence of at least one exon of the HLA locus.

The method may further include the step of analyzing the DNA sequence of the amplicon so as to provide an HLA class type for the amplicon.

The HLA allele may be a class I allele or a class II allele. The HLA allele may be selected from the group consisting of HLA-A, HLA-B, HLA-Cw, HLA-DQA1, HLA-DRB1, HLA-DRB3, HLA-DRB4, HLA-DRB5, and HLA-DQB1.

In one embodiment of the methods of the present invention, at least exon 2 of the at least one HLA allele is amplified. In this embodiment, the first portion of the first primer may be complementary to a portion of intron 1, and the first portion of the second primer may be complementary to a portion of intron 2.

In another embodiment of the methods of the present invention, at least exons 2 and 3 of the at least one HLA allele are amplified. In this embodiment, the first portion of the first primer may be complementary to a portion of intron 1, and the first portion of the second primer may be complementary to a portion of intron 3.

In another embodiment of the methods of the present invention, at least exon 4 of the at least one HLA allele is amplified.

In another embodiment of the methods of the present invention, third and fourth primers are provided for locus specific PCR amplification of at least one exon of the HLA locus other than the at least one exon in the first amplicon. The third primer includes a first portion complementary to the HLA DNA template comprising an HLA locus, and a second portion not complementary to the HLA DNA template comprising the HLA locus. The second portion of the third primer comprises a forward generic sequencing primer sequence different from the second portions of the first and second primers. The first portion is located at a 3′ end terminus of the third primer, whereas the second portion is located at a 5′ end terminus of the third primer.

The fourth primer includes a first portion that is complementary to the HLA DNA template comprising an HLA locus, and a second portion that is not complementary to the HLA DNA template comprising the HLA locus. The second portion includes a reverse generic sequencing primer sequence different from the second portions of the first, second and third primers. The first portion is located at a 3′ end terminus of the fourth primer, whereas the second portion is located at a 5′ end terminus of the fourth primer.

In one embodiment, the first portions of at least one of the third and fourth primers may comprise a nucleotide sequence of at least one of SEQ ID NOS:3-11, 24-27, 35, 45, 46, and 50-70, while the second portion of at least one of the third and fourth primers may comprise a nucleotide sequence of at least one of SEQ ID NOS:47-49. For example but not by way of limitation, at least one of the third and fourth primers may comprise a nucleotide sequence of at least one of SEQ ID NOS:1, 2, 12-23, 28-23 and 36-44.

The at least one exon of the at least one HLA allele is amplified in a locus specific fashion utilizing the third and fourth primers, thereby providing a second amplicon having two generic sequencing primer sites at the opposing terminus of the amplicon, wherein the first and second amplicons are amplified in a single amplification reaction. The second amplicon is then sequenced utilizing generic sequencing primers corresponding to the second portions of the third and fourth primers.

The present invention further includes a method for determining tissue compatibility. In the method, a tissue sample comprising at least one HLA allele is provided, and a method of typing HLA class I or class II alleles is performed as described herein above. The DNA sequence of the amplicon is then compared with at least one predetermined tissue sample.

The present invention is also related to primers that may be utilized in HLA class I or class II sequence-based typing. Such primers may comprise an isolated single stranded oligonucleotide comprising a nucleotide sequence of at least one of SEQ ID NOS:1-4, 19-24, 34-46 and 50-70.

The present invention also includes primer set of single-stranded DNA primers for determination of a HLA class I or class II type utilizing a polymerase chain reaction. The primer set comprises at least two single-stranded DNA primers of at least 15 nucleotides in length, and the primer set may comprise 4 or 5 DNA primers.

In one embodiment, the primer set is utilized for determination of a HLA class I type, and each of the at least two single-stranded DNA primers has a nucleotide sequence comprising at least one of SEQ ID NOS:1-4 and 50-53. In another embodiment, the primer set is utilized for determination of a HLA class I type, and each of the at least two single-stranded DNA primers has a nucleotide sequence comprising at least one of SEQ ID NOS:19-24 and 54-59. In yet another embodiment, the primer set is utilized for determination of a HLA class II type, and each of the at least two single-stranded DNA primers has a nucleotide sequence comprising at least one of SEQ ID NOS:34-41 and 65-70. In a further embodiment, the primer set is utilized for determination of a HLA class II type, and each of the at least two single-stranded DNA primers has a nucleotide sequence comprising at least one of SEQ ID NOS:42-46 and 60-64.

The present invention also includes kits for use in typing of HLA class I or class II alleles. Such kits may include any of the primer sets described herein above.

Other objects, features and advantages of the present invention will become apparent from the following detailed description when read in conjunction with the accompanying figures and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow diagram showing in particular the steps for determining a class I and class II HLA sequence-based type with the direct DNA sequence analysis of the present invention.

FIG. 2 is a diagram illustrating a universal sequencing amplification strategy in determining a class I HLA sequence-based type with direct DNA sequence analysis, in accordance with the methods of the present invention. The primers utilized are tagged with non-complementary sequences to the DNA template, and such tags are shown by the dotted lines. “F” and “R” indicate the use of two differing sequencing primer tags. The result is a bi-directional sequence of a single exon (such as, but not limited to, exon 2).

FIG. 3 is a diagram illustrating a universal sequencing amplification strategy in determining a class I HLA sequence-based type with direct DNA sequence analysis, in accordance with the methods of the present invention. The method of FIG. 3 is similar to the method of FIG. 2, except that the method of FIG. 3 results in an amplicon comprising two exons, such as but not limited to, exons 2 and 3, with two different sequencing primer tags flanking the two exons (i.e., a first sequencing primer tag in intron 1 and a second, different sequencing primer tag in intron 3).

FIG. 4 is a diagram illustrating a universal sequencing amplification strategy in determining a class I HLA sequence-based type with direct DNA sequence analysis, in accordance with the methods of the present invention. The method of FIG. 4 is similar to the method of FIG. 3, except that the method of FIG. 4 includes the amplification of an additional amplicon in the PCR reaction, wherein the additional amplicon comprises another exon, such as but not limited to, exon 4. One of the primers utilized for the amplification of exon 4 is tagged with non-complementary sequences to the DNA template, as illustrated by dotted line “86”, and such sequencing tag is different from the two sequencing primer tags “F” and “R” utilized for amplification of the first amplicon. The result is that at least 3 universal sequencing reactions can be carried out from the single polymerase chain reaction amplification of FIG. 4.

FIG. 5 is a diagram illustrating another universal sequencing amplification strategy in determining a class I HLA sequence-based type with direct DNA sequence analysis, in accordance with the methods of the present invention. The method of FIG. 5 is similar to the method of FIG. 2, except that only one of the two primers utilized is tagged with a non-complementary sequence to the DNA template. The amplicon so produced is then utilized for unidirectional universal sequencing of a single exon.

FIG. 6 is a diagram illustrating a universal sequencing amplification strategy in determining a class II HLA sequence-based type with direct DNA sequence analysis, in accordance with the methods of the present invention. One of the primers utilized is tagged with a non-complementary sequence to the DNA template (such tag illustrated by the dotted line), wherein the tag is utilized for a universal sequencing reaction.

FIG. 7 is a diagram illustrating a universal sequencing amplification strategy in determining a class II HLA sequence-based type with direct DNA sequence analysis, in accordance with the methods of the present invention. The primers utilized are tagged with non-complementary sequences to the DNA template, and such tags are shown by the dotted lines. “F” and “R” indicate the use of two differing sequencing primer tags. The result is a bi-directional sequence of a single exon (i.e., exon 2) of a class II allele.

FIG. 8 is a diagram illustrating a universal sequencing amplification strategy in determining a class II HLA sequence-based type with direct DNA sequence analysis, in accordance with the methods of the present invention. The method of FIG. 8 is similar to the method of FIG. 7, except that the method of FIG. 8 results in an amplicon comprising exons 2 and 3, with two different sequencing primer tags flanking exons 2 and 3 (i.e., a first sequencing primer tag in intron 1 and a second, different sequencing primer tag in intron 3).

FIG. 9 is a diagram illustrating a universal sequencing amplification strategy in determining a class II HLA sequence-based type with direct DNA sequence analysis, in accordance with the methods of the present invention. The method of FIG. 9 is similar to the method of FIG. 8, except that the method of FIG. 9 includes the amplification of an additional amplicon in the PCR reaction, wherein the additional amplicon comprises exon 4. At least one of the primers utilized for the amplification of exon 4 is tagged with non-complementary sequences to the DNA template, as illustrated by dotted line “86”, and such sequencing tag is different from the two sequencing primer tags “F” and “R” utilized for amplification of the first amplicon. The result is that at least 3 universal sequencing reactions can be carried out from the single polymerase chain reaction amplification of FIG. 9.

FIG. 10 illustrates gel electrophoresis of an amplicon comprising exon 2 of an HLA-B locus, wherein the amplicon was produced by the amplification strategy of FIG. 2.

FIG. 11 is a chromatogram illustrating bi-directional sequencing of the amplicon shown in FIG. 10.

FIG. 12 illustrates gel electrophoresis of an amplicon comprising exon 3 of a HLA-Cw locus, wherein the amplicon was produced by the amplification strategy of FIG. 2.

FIG. 13 is a chromatogram illustrating bi-directional sequencing of the amplicon shown in FIG. 12.

FIG. 14 illustrates gel electrophoresis of an amplicon comprising exons 2 and 3 of HLA-B locus, wherein the amplicon was produced by the amplification strategy of FIG. 3.

FIG. 15 is a chromatogram illustrating universal sequencing of the amplicon shown in FIG. 14 using opposing universal primers.

FIG. 16 illustrates gel electrophoresis of two amplicons, the first amplicon comprising exons 2 and 3 of HLA-B locus and the second amplicon comprising exon 4 of HLA-B locus using several patient samples as the DNA template, wherein the amplicons were produced by the amplification strategy of FIG. 4.

FIG. 17 is a chromatogram illustrating sequencing of both amplicons shown in FIG. 16, wherein M13 forward and reverse universal sequencing primers were utilized for bi-directional sequencing of the first amplicon, and primer 86 was utilized for universal sequencing of the second amplicon.

FIG. 18 illustrates gel electrophoresis of an amplicon comprising exon 4 of HLA-A locus using several patient samples as the DNA template, wherein the amplicon was produced by the amplification strategy of FIG. 5, and wherein the 86 primer was utilized as a universal sequencing tag for one of the primers utilized in the PCR reaction.

FIG. 19 is a chromatogram illustrating uni-directional universal sequencing of the amplicon shown in FIG. 18.

FIG. 20 illustrates gel electrophoresis of an amplicon comprising exon 4 of HLA-B locus using several patient samples as the DNA template, wherein the amplicon was produced by the amplification strategy of FIG. 5, and wherein the 86 primer was utilized as a universal sequencing tag for one of the primers utilized in the PCR reaction.

FIG. 21 are chromatograms illustrating uni-directional universal sequencing of the amplicon shown in FIG. 20.

FIG. 22 illustrates gel electrophoresis of an amplicon comprising exon 2 of HLA-DQA1 locus, wherein the amplicon was produced by the amplification strategy of FIG. 6, and wherein primer M13F was utilized as a universal sequencing tag for one of the primers utilized in the PCR reaction.

FIG. 23 is a chromatogram illustrating uni-directional universal sequencing of the amplicon shown in FIG. 22.

FIG. 24 illustrates gel electrophoresis of an amplicon comprising exon 2 of HLA-DQB1 locus using several patient samples as the DNA template, wherein the amplicon was produced by the amplification strategy of FIG. 6, and wherein M13 universal primer was utilized as a universal sequencing tag for one of the primers utilized in the PCR reaction.

FIG. 25 is a chromatogram illustrating uni-directional universal sequencing of the amplicon shown in FIG. 24.

FIG. 26 illustrates gel electrophoresis of an amplicon comprising exons 2 and 3 of HLA-DQA1 locus using several patient samples as the DNA template, wherein the amplicon was produced by the amplification strategy of FIG. 8, and wherein M13 universal primers were utilized as universal sequencing tags for the primers utilized in the PCR reaction.

FIG. 27 is a chromatogram illustrating universal sequencing of exon 3 of the amplicon shown in FIG. 26.

### DETAILED DESCRIPTION OF THE INVENTION

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As used herein, the term “gene” refers to a segment of DNA, composed of a transcribed region and a regulatory sequence that makes possible a transcription. The transcribed region may include an arrangement of exons alternating with introns. Exons are the coding or messenger sequence of deoxynucleotides, i.e., any intragenic region of DNA that will be expressed in mature mRNA or rRNA residues. Introns are intragenic regions of DNA that are not expressed in a mature RNA molecule. During transcription, both exons and introns are transcribed to form a primary transcript of RNA, and, following transcription, introns are excised by one or more splicing mechanism(s) to provide the mature RNA molecule, in which the coding/messenger sequence is continuous, before translation of the mature RNA molecule into polypeptide.

A “locus” is a discrete location on a chromosome that constitutes a gene. Exemplary loci are the class I MHC genes designated HLA-A, HLA-B and HLA-C; nonclassical class I genes including HLA-E, HLA-F, HLA-G, HLA-H, HLA-J and HLA-X, MIC; and class II genes such as HLA-DP, HLA-DQ and HLA-DR.

An “allele” refers to one of the multiple forms, i.e., different nucleic acid sequences, of a gene that can exist at a single gene locus on a single chromosome. One or more genetic differences can constitute an allele. Examples of HLA allele sequences are set out in Mason and Parham (Tissue Antigens, 51: 417-66 (1998)), which list HLA-A, HLA-B, and HLA-C alleles; and Marsh et al. (Hum. Immunol., 35:1 (1992)), which list HLA Class II alleles for DRA, DRB, DQA1, DQB1, DPA1, and DPB1.

A method of “identifying an HLA type” is a method that permits the determination or assignment of one or more genetically distinct HLA genetic polymorphisms. “Sequence based typing” is a particular type of HLA typing that utilizes DNA sequencing of HLA alleles for the determination or assignment of one or more genetically distinct HLA genetic polymorphisms.

The term “amplifying” refers to a reaction wherein the template nucleic acid, or portions thereof, are duplicated at least once. Unless specifically stated, “amplifying” may refer to arithmetic, logarithmic, or exponential amplification. The amplification of a nucleic acid can take place using any nucleic acid amplification system, both isothermal and thermal gradient based, including but not limited to, polymerase chain reaction (PCR), reverse-transcription-polymerase chain reaction (RT-PCR), ligase chain reaction (LCR), self-sustained sequence reaction (3SR), and transcription mediated amplifications (TMA). Typical nucleic acid amplification mixtures (e.g. PCR reaction mixture) include a nucleic acid template that is to be amplified, a nucleic acid polymerase, nucleic acid primer sequence(s), and nucleotide triphosphates, and a buffer containing all of the ion species required for the amplification reaction.

An “amplification product” is a single stranded or double stranded DNA or RNA or any other nucleic acid products of isothermal and thermal gradient amplification reactions that include PCR, TMA, 3SR, LCR, etc. The term “amplicon” is used herein to mean a population of DNA molecules that have been produced by amplification, such as but not limited to, by PCR. The terms “amplification product” and “amplicon” are used therein interchangeably.

The phrase “template nucleic acid” refers to a nucleic acid polymer that is sought to be copied or amplified. The “template nucleic acid(s)” can be isolated or purified from a cell, tissue, animal, etc. Alternatively, the “template nucleic acid(s)” can be contained in a lysate of a cell, tissue, animal, etc. The template nucleic acid can contain genomic DNA, cDNA, plasmid DNA, etc. In addition, the “template nucleic acid” may be an amplicon or PCR product from a prior amplification reaction.

The term “oligonucleotide” as used herein refers to a molecule having two or more deoxyribonucleotides or ribonucleotides, preferably more than three deoxyribonucleotides. The exact number of nucleotides in the molecule will depend on the function of the specific oligonucleotide molecule. As used herein the term “primer” refers to a single stranded DNA oligonucleotide sequence that may be produced synthetically and which is capable of acting as (1) a point of initiation for synthesis of a primer extension product which is complementary to a nucleic acid strand to be copied; or (2) a point of initiation for sequencing a DNA molecule. In the case of primers intended for use in synthesizing cDNA or amplifying cDNA or genomic DNA molecules by polymerase chain reaction products, the length and sequence of the primer must be sufficient to prime the synthesis of extension products in the presence of a polymerization enzyme. In one embodiment, the length of the primer may be from about 5-50 nucleotides, such as from about 5-20 nucleotides. Specific length and sequence of the primer will depend on complexity of required DNA or RNA target templates, as well as reaction conditions. When nested primers are used for sequencing, the number of base pairs separating the amplification and sequencing primers on the DNA template are also important considerations.

An “HLA allele-specific” primer is an oligonucleotide that hybridizes to nucleic acid sequence variations that define or partially define that particular HLA allele.

An “HLA locus-specific” primer is an oligonucleotide that permits the amplification of a HLA locus sequence orthat can hybridize specifically to an HLA locus.

A “forward primer” and a “reverse primer” constitute a pair of primers that can bind to a template nucleic acid and, under proper amplification conditions, produce an amplification product. If the forward primer is binding to the sense strand, then the reverse primer is binding to antisense strand. Alternatively, if the forward primer is binding to the antisense strand, then the reverse primer is binding to the sense strand. However, it is to be understood that the forward and reverse primers can bind to either strand as long as the other reverse or forward primer binds to the opposite strand.

The terms “universal sequencing primer”, “generic sequencing primer”, and variants thereof, as used herein, will be understood to include any sequence that is not found in a DNA template of a locus to be amplified and that can be utilized as a primer for DNA sequencing, that is, the primer has a proper annealing temperature for the sequencing reaction. Examples of universal sequencing primers are well known in the art and are fully within the scope of knowledge of a person having ordinary skill in the art. Particular examples that may be utilized in accordance with the invention include, but are not limited to, M13 Forward and Reverse, T3, T7, SP6, BGH, pGEX Forward and Reverse, lambda gt10 Forward and Reverse, various pBR322 primers, and the like. However, while known universal sequencing primers such as M13 forward and reverse are believed by the prior art to be advantageous because they do not match any sequence in the human genome, the present invention discloses that any sequence may be utilized as a “universal sequencing primer” that is attached to the 5′ end of a primer (wherein the 3′ end of the primer is sequence specific), as long as such sequence does not correspond to a sequence in the locus to be amplified and the resulting primer has a proper annealing temperature for the sequencing reaction. The present invention demonstrates that even though the “universal sequencing primer” or “generic sequencing primer” may correspond to another sequence present in the genomic DNA, such primer will not result in amplification of such undesired DNA, because polymerases require structural conformation at the 3′ end of the primer in order to extend the primer.

The present invention discloses a method for typing HLA class I alleles in order to determine the particular type of a sample, for example but not by way of limitation, the particular HLA-A, -B, or -Cw type of a sample. The method comprises at least one amplification step to amplify, purify and separate at least one exon of class I in a locus specific manner. In particular, the method comprises the steps of: (a) amplifying HLA class I alleles in a locus-specific manner so as to provide at least one amplicon, wherein the amplicon comprises at least one amplified exon of the HLA allele, and wherein the at least one amplicon is provided with two opposing different universal sequencing primer sites attached to the opposing terminus of the at least one amplicon; and (b) performing bi-directional DNA sequencing of the at least one amplicon utilizing the two different opposing universal sequencing primers. The universal sequencing primer sites may be any sequence that is not complementary to the locus being amplified. In addition, the amplicon produced in (a) has been demonstrated herein to serve as an adequate DNA template for DNA sequencing.

In one embodiment, the method further comprises the step of analyzing the DNA sequence of the at least one amplicon so as to provide an HLA class I type for the at least one amplicon.

The present invention also discloses a method for typing HLA class II alleles in order to determine the particular type of a sample, for example but not by way of limitation, the particular HLA-DRB1, -DRB3, -DRB4, -DRB5, -DQB1, -DQA1 or -DPB1 type of a sample. The method comprises at least one amplification step to amplify, purify and separate at least one exon of class II in a locus specific manner. In particular, the method comprises the steps of: (a) amplifying HLA class II alleles in a locus-specific manner so as to provide at least one amplicon, wherein the amplicon comprises at least one amplified exon of the HLA allele, and wherein the at least one amplicon is provided with at least one universal sequencing primer site attached to the at least one amplicon; and (b) performing DNA sequencing of the at least one amplicon utilizing the universal sequencing primer. The universal sequencing primer site may be any sequence that is not complementary to the locus being amplified. In addition, the amplicon produced in (a) has been demonstrated herein to serve as an adequate DNA template for DNA sequencing, without requiring any additional isolation or purification steps.

In one embodiment, the method further comprises the step of analyzing the DNA sequence of the at least one amplicon so as to provide an HLA class II type for the at least one amplicon.

In an alternative embodiment, the amplicon produced in the method of typing HLA class II alleles described herein above may be provided with two opposing different universal sequencing primer sites attached to the opposing terminus of the at least one amplicon, and bi-directional DNA sequencing of the at least one amplicon may be performed utilizing the two different opposing universal sequencing primers.

Any of the methods of typing class I or class II alleles described herein above may further include amplification of a second amplicon comprising at least one exon other than the at least one exon present in the first amplicon, wherein the first and second amplicons are amplified in a single PCR reaction. This second amplicon is also provided with at least one universal sequencing primer site attached thereto, and may be provided with two opposing different universal sequencing primer sites attached to the opposing terminus of the second amplicon. In this manner, three or more universal sequencing reactions can be carried out from a single polymerase chain reaction.

Each of the primers utilized in the methods of the present invention comprise: (a) a locus specific sequence that corresponds to a portion of a locus-specific sequence, wherein the sequence is complementary to the HLA locus DNA template and flanks an exon desired for amplication; and (b) a universal or generic sequencing primer sequence that does not correspond to any portion of the locus sequence to be amplified. The locus specific sequence is present at a 3′ end terminus of the primer, whereas the universal sequencing primer sequence is located at a 5′ end terminus of the primer. For each PCR amplification, the two primers utilized for amplification of an amplicon must have different universal sequencing primer sequences. If two or more amplicons are produced for a single PCR reaction, all of the primers utilized in the single PCR reaction must have different universal sequencing primer sequences. That is, for amplification of a single amplicon, two different universal sequencing primer sequences will be present; when two amplicons are amplified, four different universal sequencing primer sequences are used.

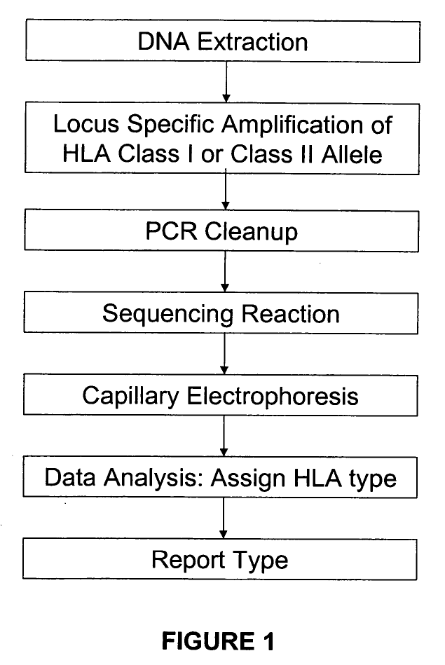

FIG. 1 illustrates an overview of the methods for determining HLA class I and class II types via locus specific amplification. The methods of the presently claimed and disclosed invention begin by obtaining a sample, such as but not limited to, whole blood, tumor cells, sperm, hair follicles, or any other nucleated tissue sample. Once a sample is obtained, the first step is to treat the tissue sample so as to obtain nucleic acids for amplification.

The purpose of the PCR reaction is to generate PCR products which are generally 350-1100 nucleotides in length (although the PCR product may be any length so long as it functions as described herein), and to produce PCR products containing opposing universal sequencing primer sites. For sequence based typing of HLA class I and class II alleles, the presently disclosed and claimed invention utilizes two DNA sequencing reactions per amplicon, with the forward sequencing primer site built onto the PCR primer which opposes the reverse sequencing primer site, so that universal sequencing primers flank the exon(s) being sequenced. This technique is referred to as bi-directional universal sequencing of an HLA allele. One advantage of the presently claimed and disclosed invention is the ability to sequence all class I and class II reactions with the same small set of universal sequencing primers, as compared to the prior art methods of utilizing sets of sequencing primers for each and every HLA locus.

The amplicons produced by the PCR reactions of the presently claimed and disclosed invention are demonstrated herein to serve as an adequate template for DNA sequencing. For example but not by way of limitation, the amplicons may be utilized in end terminus cycle sequencing, in which single stranded fragments of the amplicon are fluorescently labeled during cycle sequencing and separated by a capillary sequencer.

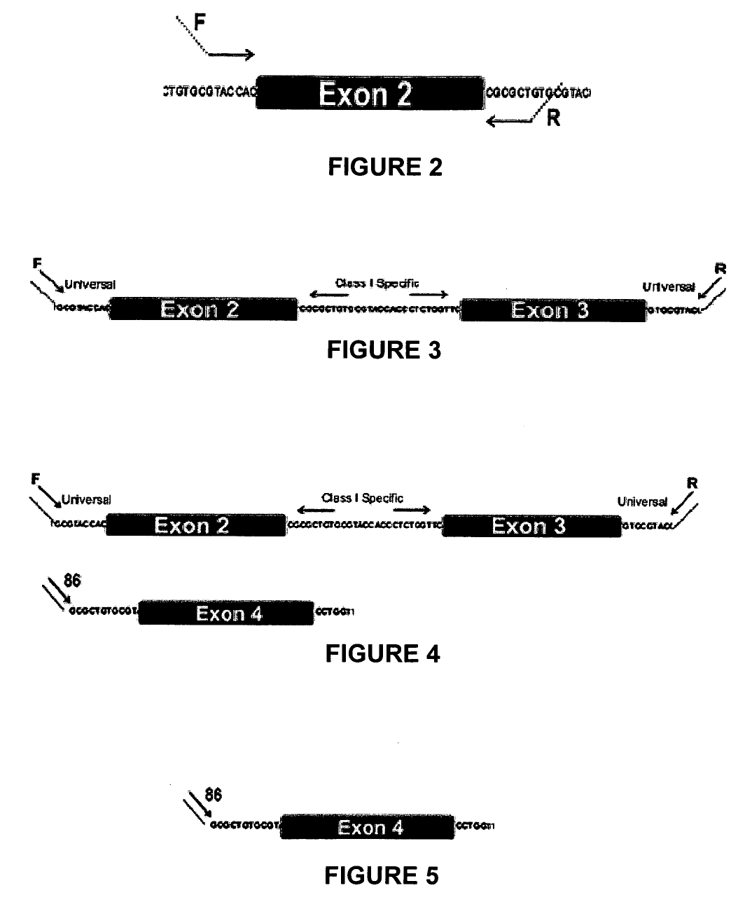

FIGS. 2-5 illustrate amplification strategies utilized in HLA Class I sequence-based typing methods of the presently claimed and disclosed invention. However, it is to be understood that such particular amplification strategies are provided for the purposes of illustration only, and that such amplification strategies are in no way considered limiting of the invention. For example, the amplification strategies of the present invention are not limited to the amplification of particular exons, placement of primer sequences and/or universal sequencing tags, and use of tagged sequencing primers and/or locus-specific sequencing primers shown in FIGS. 2-5. Rather, the scope of the presently claimed and disclosed invention includes the use of any amplification strategy that produces an amplicon that functions in accordance with the present invention.

With reference now to FIG. 2, such diagram illustrates an amplification strategy utilized in determining a class I HLA sequence-based type with direct DNA sequence analysis, in accordance with the methods of the present invention. In FIG. 2, one exon of a HLA class I allele is amplified using two primers having sequencing tags disposed thereon. FIG. 2 illustrates this exon as being exon 2; however, the use of exon 2 is simply for illustration purposes herein and is not to be regarded as limiting. When the exon to be amplified is exon 2, the first primer comprises a portion that is complementary to a portion of intron 1 flanking exon 2 (illustrated as a straight line above the sequence of intron 1), and a portion that does not correspond to any sequence in the DNA template of the particular HLA locus being amplified (illustrated by a dotted, diagonal line and the letter “F”). In this instance, the second primer comprises a portion that is complementary to a portion of intron 2 flanking exon 2 (illustrated as a straight line below the sequence of intron 2), and a portion that does not correspond to any sequence in the DNA template of the HLA locus (illustrated by a dotted, diagonal line and the letter “R”). “F” and “R” generically refer to Forward and Reverse primers (for the purposes of illustrating direction of nucleotide synthesis only), and also indicate that the two sequencing primer tags are not the same but rather are different sequences. The amplicon produced by the amplification strategy of FIG. 2 may be utilized for bi-directional sequence of a single exon (such as, but not limited to, exon 2).

FIG. 3 illustrates another amplification strategy useful in determining a class I HLA sequence-based type in accordance with the methods of the present invention. The method of FIG. 3 is similar to the method of FIG. 2, except that the amplicon produced by the method of FIG. 3 comprises at least two exons. FIG. 3 illustrates these exons as being exons 2 and 3; however, the use of exons 2 and 3 is simply for illustration purposes herein and is not to be regarded as limiting. The amplicon produced by the method of FIG. 3 comprises two different opposing sequencing primer tags that flank the at least two exons; these tags are produced by utilizing primers as described above in relation to FIG. 2, except that when the amplicon produced comprises exons 2 and 3, the first primer comprises a portion that is complementary to a portion of intron 1 flanking exon 2, and the second primer comprises a portion that is complementary to a portion of intron 3 flanking exon 3. The amplicon produced by the amplification strategy of FIG. 3 may be utilized for bi-directional sequence of two or more exons (such as, but not limited to, exons 2 and 3). Depending on the size of the amplicon, it may be desired to utilize locus-specific primers to obtain the entire bi-directional sequence of the amplicon. FIG. 3 illustrates the use of two locus-specific sequencing primers that are each complementary to a portion of intron 2; however, it is to be understood that such locus-specific sequencing primers may be complementary to any portion of the amplicon produced by the method of FIG. 3, including sequences of the introns as well as exons present, and therefore the placement of the locus-specific sequencing primers in FIG. 3 is simply for illustration purposes only and is not to be regarded as limiting.

In addition, while only some of the Figures illustrate the use of locus-specific sequencing primers herein, it is to be understood that any of the methods of the present invention may include the use of one or more locus-specific sequencing primers in combination with one or more universal or generic sequencing primers in direct DNA sequencing.

FIG. 4 illustrates another universal sequencing amplification strategy for use in determining a class I HLA sequence-based type with direct DNA sequence analysis, in accordance with the methods of the present invention. In FIG. 4, a first amplicon similar to that produced in FIG. 2 or 3 is produced in the PCR reaction (for illustration purposes only, the amplicon of FIG. 3 is shown as the first amplicon). In addition, the method of FIG. 4 includes the amplification of a second amplicon in the single PCR reaction, wherein the additional amplicon comprises at least one additional exon (FIG. 4 illustrates this at least one additional exon as being exon 4 for illustration purposes only). At least one of the primers utilized for the amplification of the second amplicon is tagged with non-complementary sequences to the DNA template of the HLA locus, as illustrated by dotted line “86”; the sequencing tag “86” is different from the two sequencing primer tags “F” and “R” utilized for amplification of the first amplicon. The result is that two amplicons are produced in the single polymerase chain reaction amplification step, and at least 3 universal sequencing reactions can be carried out from the single polymerase chain reaction amplification of FIG. 4.

FIG. 5 illustrates yet another amplification strategy for determining class I HLA type with direct DNA sequence analysis, in accordance with the present invention. The method includes amplifying an HLA class I allele in a locus-specific manner so as to provide at least one amplicon, wherein the amplicon comprises at least one exon of the HLA class I allele (exon 4 is shown for illustration purposes only). In FIG. 5, the amplicon is provided with a universal sequencing primer tag (illustrated by the dotted line “86”) attached to one end thereof. The other end of the amplicon will not contain a universal sequencing primer site, and therefore bi-directional sequencing of the amplicon produced as shown in FIG. 5 will require at least one locus-specific sequencing primer to sequence exon 4 in both directions.

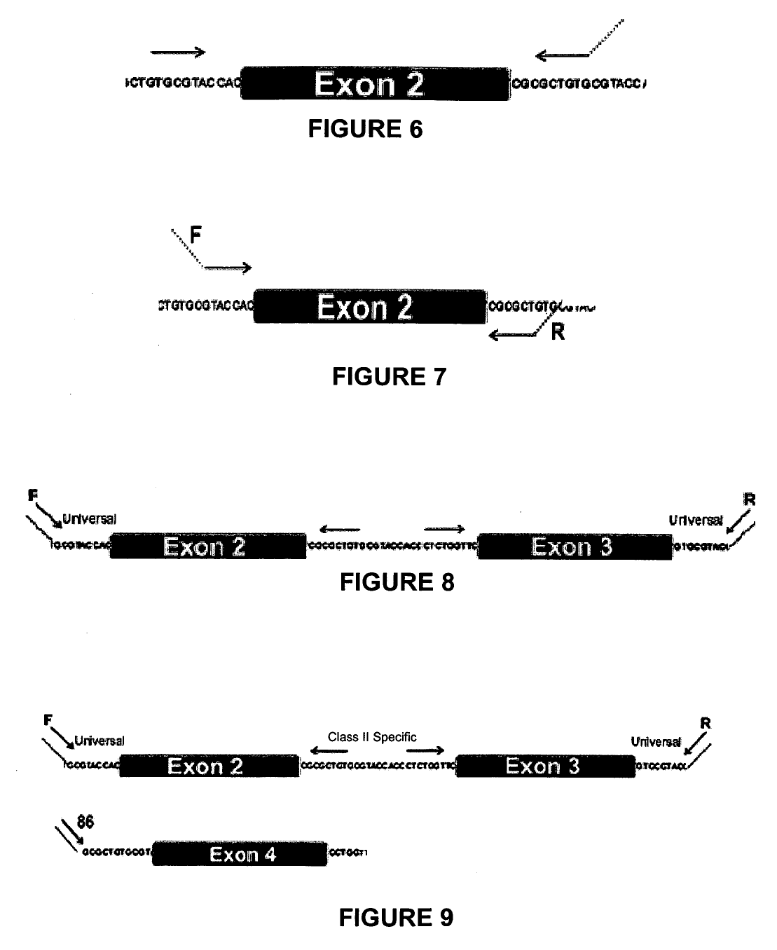

FIGS. 6-9 illustrate amplification strategies utilized in HLA Class II sequence-based typing methods of the presently claimed and disclosed invention. However, it is to be understood that such particular amplification strategies are provided for the purposes of illustration only, and that such amplification strategies are in no way considered limiting of the invention. For example, the amplification strategies of the present invention are not limited to the amplification of particular exons, placement of primer sequences and/or universal sequencing tags, and use of tagged sequencing primers and/or locus-specific sequencing primers shown in FIGS. 6-9. Rather, the scope of the presently claimed and disclosed invention includes the use of any amplification strategy that produces an amplicon that functions in accordance with the present invention.

The amplification strategies of FIGS. 6-9 are utilized in methods for determining class II HLAtype, such as but not limited to, HLA-DRB1, -DRB345, -DQB1, -DQA1, and -DPB1. Such methods specifically amplify, with a single locus-specific PCR reaction, PCR products containing one or more universal sequencing primer sites. Sequencing is carried out by a universal primer annealing to the attached end terminus sequencing site of the amplicon tagged during amplification by primer incorporation of such a site on the end of the primer.

FIG. 6 illustrates an amplification strategy utilized in determining a class II HLA sequence-based type with direct DNA sequence analysis, in accordance with the methods of the present invention. The method includes amplifying an HLA class II allele in a locus specific manner so as to provide at least one amplicon, wherein the amplicon comprises at least one amplified exon of the HLA allele. In FIG. 6, the amplicon is provided with a universal sequencing primer tag attached to one end thereof (illustrated as a dotted, diagonal line attached to the straight line representing the portion of the primer that is complementary to the DNA template). The other end of the amplicon will not contain a universal sequencing primer site, and therefore bi-directional sequencing of the amplicon produced as shown in FIG. 6 will require at least one locus-specific sequencing primer to sequence the exon(s) in both directions. The method of FIG. 6 may be utilized, for example but not by way of limitation, to obtain the sequence of exon 2 of HLA-DRB1, DRB-3, DRB-4, or DRB-5, as there is much variability present in the sequence of intron 1 for the various alleles of these loci.

FIG. 7 illustrates an amplification strategy utilized in determining a class II HLA sequence-based type with direct DNA sequence analysis, in accordance with the methods of the present invention. In FIG. 7, bi-directional sequencing of HLA class II is performed as described herein above, and in a similar manner to that described herein above for the amplification strategy utilized in the method of HLA class I typing of FIG. 2. Briefly, the method of FIG. 7 includes amplifying HLA class II allele in a locus-specific manner so as to provide at least one amplicon. The amplicon comprises at least one amplified exon of the HLA class II allele, and the at least one amplicon is provided with two opposing different universal sequencing primer sites attached to the opposing terminus of the at least one amplicon. The universal sequencing primer sites may be any sequence that is not present in the locus to be amplified. FIG. 7 illustrates the amplicon as comprising exon 2; however, it is to be understood that any exon of an HLA class II locus may be amplified and sequenced in accordance with the methods of the present invention, and therefore, the use of exon 2 is not to be regarded as limiting.

The method of the present invention also includes the amplification of more than one HLA class II exon, as shown in FIG. 8. In FIG. 8, a single amplicon is produced in a single PCR reaction, in a similar manner as described herein above with reference to FIG. 3, and the single amplicon is provided with universal sequencing tags on the opposing ends thereof to facilitate bi-directional sequencing of two or more exons (exons 2 and 3 are shown and described herein for purposes of illustration only). However, depending on the lengths of exons 2 and 3 and intron 2, entire sequence of exons 2 and 3 in both directions may not be obtainable simply using the universal sequencing primers to the universal sequencing tags located at opposing ends of the amplicon. Therefore, it may be necessary to utilize locus-specific sequencing primers as well. FIG. 8 depicts two locus-specific sequencing primers that correspond to sequences in intron 2; however, it is to be understood that such sequencing primers may correspond to any intronic or exonic sequence, and therefore the use of intronic primers in FIG. 8 is simply for illustration purposes only.

In another alternative, the allelic groups of HLA-DQA1 class II locus contain a deletion in exon 2 at codon 56, and therefore identification of HLA-DQA1 alleles requires a secondary amplification step to separate the allelic groups containing codon 56 deletions and incorporate another universal primer site. Heterozygous DNA sequencing requires a precise distance from the primer end to align heterozygous strands up for determining an exact type. The identification of HLA-DQA1 locus requires separation for samples with both groups of alleles to prevent undistinguishable sequences from the point of the codon deletion of heterozygous amplified template to the end of the sequencing.

FIG. 9 illustrates yet another amplification strategy for methods of determining HLA class II type, wherein such amplification strategy is similar to that described herein above with relation to FIG. 4. Briefly, a first amplicon similar to that produced in FIG. 6, 7 or 8 is produced in the PCR reaction, and a second amplicon comprising at least one additional exon, for example but not by way of limitation, exon 4, is also produced in the PCR reaction. At least one of the primers utilized for the amplification of the second amplicon is tagged with non-complementary sequences to the DNA template of the HLA locus, as illustrated by dotted line “86”; the sequencing tag “86” is different from the two sequencing primer tags “F” and “R” utilized for amplification of the first amplicon. The result is that two amplicons are produced in the single polymerase chain reaction amplification step, and at least 3 universal sequencing reactions can be carried out from the single polymerase chain reaction amplification of FIG. 9.

For example, a method to amplify exons 2 and 3 of HLA-DQB1 is not ideal due to the extremely long intron 2 sequence of this locus. Therefore, for HLA-DQB1, it may be desirable to include two additional primers for locus-specific amplification of exon 3 in the same PCR reaction in which exon 2 is amplified, thereby producing two amplicons (one for exon 2, as described above, and a second amplicon for exon 3). One or both of the two additional primers may be provided with universal sequencing tags attached thereto, so that the second amplicon comprises exon 3 framed by one or two universal sequencing tags.

Once an amplicon is produced as shown in FIGS. 2-9, the at least one amplicon with the two different opposing universal sequencing primers (except as shown in FIGS. 5 and 6, where only one universal sequencing primer is utilized) is directly subjected to DNA sequencing without requiring any purification/isolation steps.

The amplicon produced by PCR is tagged during amplification by incorporation of a primer having a sequencing site on an end thereof, and sequencing is carried out by a universal primer annealing to the attached end terminus sequencing site of the amplicon. Read lengths in which class I and class II HLA heterozygous positions are resolved generally range from 300-350 nucleotides from either direction, with heterozygous positions proximal to the sequencing primers best resolved. Following this scheme, positions of heterozygosity immediately adjacent to each primer are most clearly resolved, while it becomes more difficult to resolve heterozygous positions as reads progress beyond 350 bases from each sequencing primer. Before determining a class I or class II type, the positions of heterozygosity should be clearly resolved beyond 200 nucleotides for both sequencing reads in each exon.

The software then compares all possible combinations of HLA-A, -B, or -C alleles in an existing HLA locus specific database to the assembled sequencing data. The software then sequentially ranks the pairs of HLA class I alleles which best fit the sequence data and lists the number of positions not consistent with the best fit pair of alleles in the class I database.

The software is arranged so that ambiguities and questioned calls are flagged, and flagged calls are readily viewed in the chromatograms which are present in the software window. The technique described herein is not foolproof in that <30% of the HLA-A and HLA-B heterozygous combinations of alleles are non-unique through exons 2 and 3 and cannot be resolved with this SBT technique. For example, the current method cannot distinguish a B*1501/B*4008 DNA sequence combination from a B*1508/B*4002 individual, such that a second class I HLA typing step (i.e. PCR-SSP) or a third sequencing reaction through exons 4-8 will sometimes be required. However, this is true of any technique that types only exons 2 and 3. One of ordinary skill in the art would appreciate that the level of resolution obtained is beyond that of other methodologies.

The scope of the present invention includes polymerase chain reaction primers that include degenerate bases to hybridize to all of the alleles comprising a locus. These primers may generally have a size range of from 20-43 nucleotides in length, with DNA template complementary portion of the primer having a size range of about 17 to about 23 nucleotides, and the universal sequencing tag portion of the primer having a size range of about 15 to about 20 nucleotides.

The class I and class II SBT methods of the present invention are robust, cost-efficient, quick, and require substantially less sequencing mixes when compared to the current SBT methods of the prior art. Since the methods of the present invention do not require locus specific sequencing primers, the methods have profound advantages in validation and quality control of sequencing primers, sequencing mixes, packaging, and protocol development.

Examples are provided hereinbelow. However, the present invention is to be understood to not be limited in its application to the specific experimentation, results and laboratory procedures. Rather, the Examples are simply provided as one of various embodiments and is meant to be exemplary, not exhaustive.

### Materials and Methods for the Examples

The first step for each example involved PCR amplification of the desired amplicon. As the amplicon varied for each example, the particular primers will be described in detail under each example.

The primers listed in Table 1 correspond to the bases which are the same across the introns and are indicated as a single base (A, C, G, T). However, it is to be understood that the primers utilized in accordance with the present invention may also be provided with alternative bases when bases are variable across the locus-specific sequence of the different alleles.

Specific buffers and reagents used for this PCR have been previously described in the literature, are widely known in the art and commercially available, and one of ordinary skill in the art would appreciate that any PCR buffer is contemplated for use provided that it functions in accordance with the methods of the present invention.

Once the sample has been treated, it is combined with two amplification primers and amplified, for example using PCR amplification. The basic process of PCR amplification is known, for example from U.S. Pat. Nos. 4,683,202 and 4,683,195 (both issued to Mullis et al., on Jul. 28, 1987), which are specifically incorporated herein by reference. In PCR amplification, two amplification primers are used, each of which hybridizes to a different one of the two strands of a DNA duplex. Multiple cycles of primer extension and denaturation are used to produce additional copies of DNA extending from the position of one primer to the position of the other. In this way, the number of copies of the genetic material positioned between the two primer binding sites is increased.

In the present invention, amplification is preferably performed using at least one locus-specific primer which specifically hybridizes to a portion of the areas surrounding a targeted exon(s) of a HLA locus, for example but not by way of limitation, introns 1, 2 or 3, or exon 2 for nested amplification of DQA1. As used in the present invention, the primers which “specifically hybridize” to the DNA template are primers which permit locus-specific amplification by having a sequence which is exactly complementary to the expected sequence of a portion of the intron and/or exon so that binding and amplification can occur, but which is not complementary to a region on any of the other HLA genes. Degenerate bases can be introduced in the primer sequences where alternative bases occur among alleles. It will be understood that locus-specific primers within the scope of this invention need not be complementary to a totally unique sequence within the human genome, provided that both members of the primer pair used in amplification do not bind to the same gene outside the gene of interest.

In addition to primers binding to the non-coding strand, it will be appreciated that complementary primers which bind to the corresponding portions of the coding strand could be used with a compatible second primer. The use of complementary locus-specific primers also falls within the scope of the present invention.

While PCR amplification is the preferred approach to amplification of the treated sample, other techniques which use oligonucleotide primers to define a region of DNA to be amplified can be used as well. Examples of such techniques have been described herein above, and it will be understood that any method of amplification resulting in an amplicon that can be subjected to direct DNA sequencing may be utilized in accordance with the present invention.

Following PCR, the PCR product is purified to remove dNTP's and unincorporated primers. This purification step may be performed by any method known in the art, and kits for purification of PCR products are commercially available. Such kits may utilize enzymatic digest, column filtration, magnetic beads, or other purification methods known in the art.

Once the PCR product is purified, it is subjected to cycle sequencing. Methods of performing cycle sequencing reactions are known in the art, and kits for performing cycle sequencing reactions are commercially available. Following cycle sequencing, removal of unincorporated fluorescent dyes can be performed by any method known in the art, such as but not limited to, ethanol precipitation or filtration.

Provided herein after are the particular materials and methods of the examples; however, such materials and methods are in no way to be considered limiting of the invention, and are simply provided as one possible example of how the methods of the present invention may be carried out.

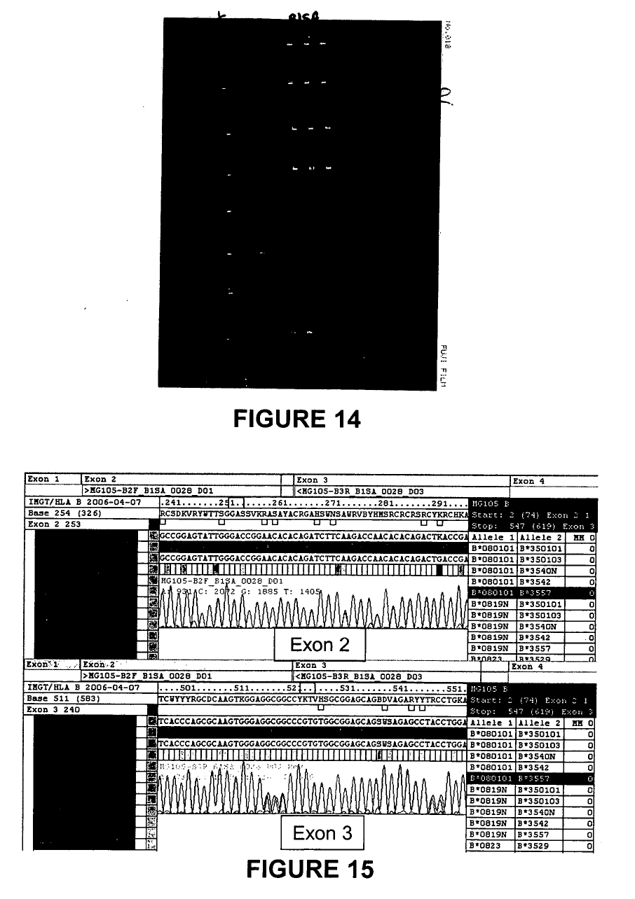

With the exception of Examples 1 and 2, all of the PCR reactions were carried out as follows: 200 ng of genomic DNA was combined with 0.9× Roche FastTaq PCR Master Mix and 6 picomoles of primers in their respective mix (A4, B1, B2, B3, B4, B1SA, C1, C2, C3, DQA2, DQA23, or DQB1; see Table 1) in a single reaction. The thermal cycler conditions were as follows: 8 minutes at 96° C., followed by 35 cycles of 20 seconds at 94° C., 30 seconds at 62° C. and 75 seconds at 72° C. Amplification was confirmed by electrophoresis on a 2% agarose gel, as shown in FIGS. 14, 16, 18, 20, 22, 24, and 26. The PCR product was prepared for PCR purification by diluting 1 to 20.

For Examples 1 and 2, a primary PCR was performed, followed by a secondary nested PCR. 20 μl PCR reaction was prepared by combining 400 ng genomic DNA, 0.06 mM dNTP, 0.05 μl mM MgCl₂, 3.3 mM Tris-HCl, 16.7 mM KCl, 4 picomoles primers (B1 primer set for Example 1 and C1 primer set for Example 2), and 0.5 U/μl Taq polymerase. The thermal cycler conditions for the primary PCR were as follows: 2 minutes at 96° C., followed by 35 cycles of 30 seconds at 94° C., 45 seconds at 56° C. for Example 1 or 54° C. for Example 2, and 1 minute at 72° C. Amplification of the primary PCR product was confirmed by electrophoresis on a 2% agarose gel.

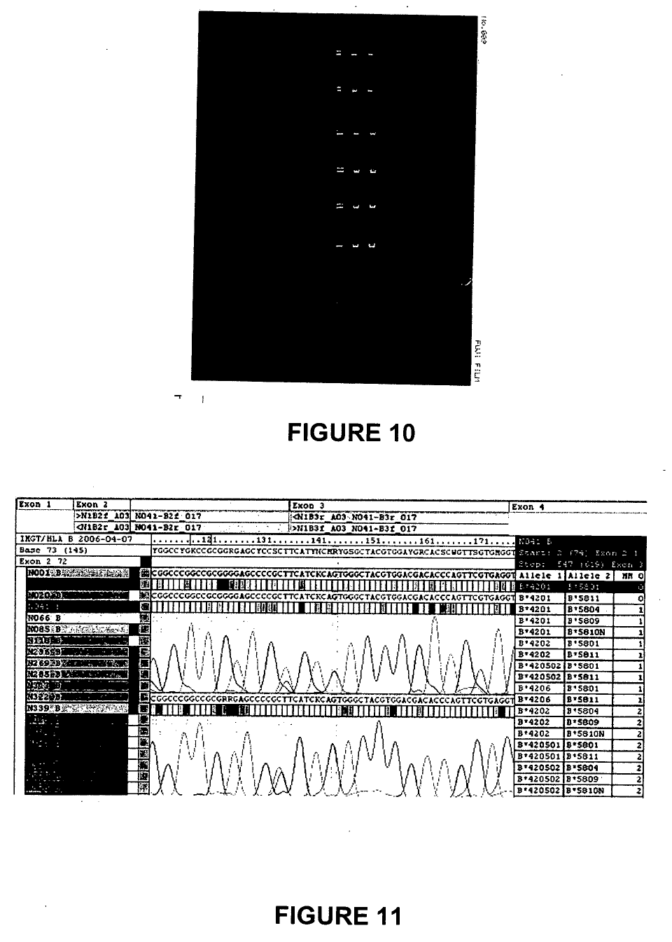

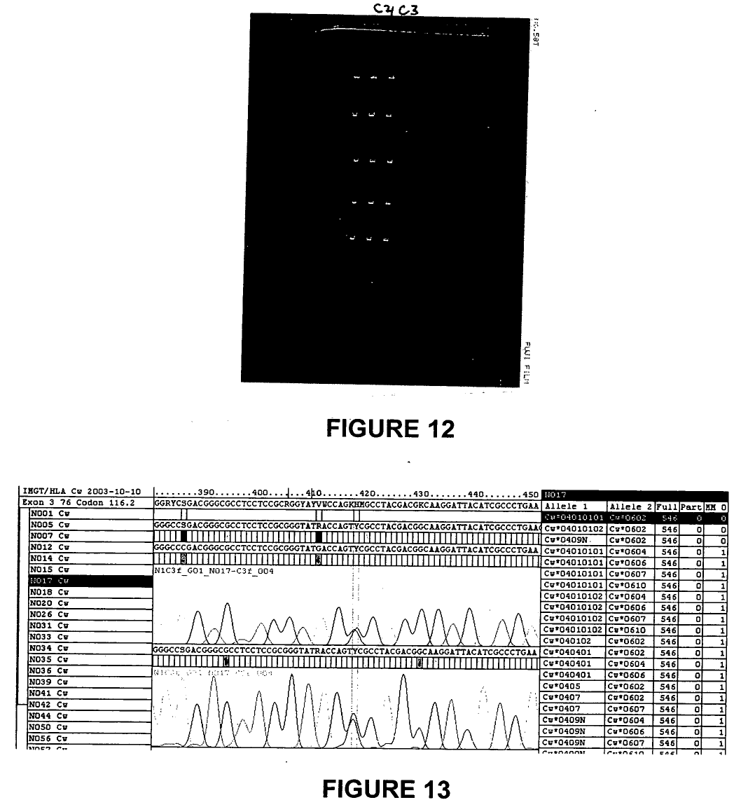

The secondary nested PCR reaction of Examples 1 and 2 was performed by combining 2 μl of 1 to 100 dilute primary PCR product, 0.06 mM dNTP, 0.5 μl MgCl₂, 3.3 mM Tris-HCl, 16.7 mM KCl, 4 picomoles primers (B2 and B3 primer sets for Example 1 and C2 and C3 primer sets for Example 2), and 0.5 U/μl Taq polymerase. The thermal cycler conditions for the primary PCR were as follows: 2 minutes at 95° C., followed by 30 cycles of 30 seconds at 95° C., 30 seconds at 56° C. for Example 1 or 54° C. for Example 2, and 45 seconds at 72° C. Amplification of the secondary, nested PCR product was confirmed by electrophoresis on a 2% agarose gel, as shown in FIGS. 10 and 12.

The second step in each example involved PCR Purification to remove unincorporated primers and dNTPs prior to the sequencing reaction. The PCR purification reaction was prepared by adding 4 μl of the diluted PCR product, 0.4 μl Water, 0.6 μl 10×SAP (Shrimp Alkaline Phospatase) Buffer, 0.5 μl Shrimp Alkaline Phosphate, and 0.5 μl Exonuclease I. The react-ion was incubated for 30 minutes at 37° C. followed by 15 minutes at 80° C.

The third step in each example involved a cycle sequencing reaction. Basic procedures for performing nucleic acid sequencing in this manner are well known in the art, and commercial instruments are available for this purpose. Thus, sequencing is a routine procedure provided that amplified DNA and suitable primers are available. In this case, the same primers used to amplify the DNA can be used as sequencing primers. In the particular instance of the examples, a cycle sequencing reaction (Big Dye Cycle Sequencing Terminator Kit, Applied Biosystems) was prepared as follows: 1 μl 5× Big Dye Buffer (Applied Biosystems), 3.2 picomoles universal primer (Table 1), and 1 μl Big Dye V3.1 were combined and brought to a 10 μl volume with water in the purified PCR product reaction tube. The reaction was run on a thermal cycler with the following conditions: 96° C. for 2 minutes, followed by 50 cycles of 20 seconds at 96° C., 30 seconds at 50° C., and 2 minutes at 72° C.

The fourth step in each example involved sequencing cleanup. 30 μl of 95% ethanol, 0.12M Sodium Acetate was added to each sequencing reaction, followed by centrifugation at 3000 rpm for 30 minutes at 4° C. The sample was then subjected to pulse centrifugation up to 500 rpm inverted on a paper towel square. 30 μl of 71% ethanol was added, followed by centrifugation at 3000 rpm for 15 minutes at 4° C. The sample was again subjected to pulse centrifugation up to 600 rpm inverted on a paper towel square, followed by air drying for 20 minutes. After the ethanol was completely evaporated, the precipitant was resuspended in 20 μl of water.

The fifth step in each example involved capillary electrophoresis. The sequencing fragments were separated by capillary electrophoresis on an optimized Applied Biosystems 3100 or 3700 series DNA sequencer.

For interpretation of the data obtained in each example, the trace files from the DNA sequencer were imported into commercially available HLA specific software (Assign by Conexio Genomics of David, Australia). The software consists of an internal base calling code and alignment against an internal data base of known HLA allele library based on the HLA IMGT Database (www.ebi.ac.uk/imgt/hla).

TABLE 1

SEQ

| | |
|---|---|
| Mix | ID |

| | | | | | |
|---|---|---|---|---|---|
| Locus | Number | Primer Name | Location | Primer Sequence 5′ to 3′ | NO: |

| | | | | | |
|---|---|---|---|---|---|
| A | A4 | A1e4-1-86 | Intron 3> | CACTGTGAAGCTCTCCAGCTG | 1 |
| | | | | GTGGAGTGTCCCATG | |
| A | A4 | A1e4-2-86 | Intron 3> | CACTGTGAAGCTCTCCAGCTG | 2 |
| | | | | GAGGAGTGTCCCATG | |
| A | A4 | A1e4-3 | <Intron 4 | GGTCTCCAGAGAGGCTCCTG | 3 |
| A | A4 | A1e4-4 | <Intron 4 | AAGGTCAGAGAGGCTCCTGC | 4 |
| B | B1 | 3′BI-31 | <Intron 3 | AGCCCATCCCCGCCGACCTAT | 5 |
| B | B1 | 3′BI-32 | <Intron 3 | AGGCCATCCCCGCCGACCTAT | 6 |
| B | B1 | 3′BI-33 | <Intron 3 | AGGCCATCCCCGGCGACCTAT | 7 |
| B | B1 | 3′BI-34 | <Intron 3 | AGGCCATCCCGGGCGATCTAT | 8 |
| B | B1 | 5′BI11 | Intron 1> | GAGGAGAGAGGGGACCGCAG | 9 |
| B | B1 | 5′BI12 | Intron 1> | GAGGAGCAAGGGGACCGCAG | 10 |
| B | B1 | 5′BI13 | Intron1> | GAGGAGCGAGGGGACCGCAG | 11 |
| B | B2 | 5′I1E2B-U | Intron 1> | GTAAAACGACGGCCAGTGCGC | 12 |
| | | | | CGGGAGGAGGGTC | |
| B | B2 | 3′I2E2ABC-1UR | <Intron 2 | CAGGAAACAGCTATGACCAGT | 13 |
| | | | | CGTGACCTGCGCCCC | |
| B | B2 | 3′I2E2ABC-2UR | <Intron 2 | CAGGAAACAGCTATGACCAGT | 14 |
| | | | | CCTGACCTGCGCCCC | |
| B | B3 | 3′bcI3-UR | <Intron 3 | CAGGAAACAGCTATGACCAAG | 15 |
| | | | | ATGGGGAAGGCTCCCCACT | |
| B | B3 | 5′I2E3B-U1 | Intron 2> | GTAAAACGACGGCCAGTGGGG | 16 |
| | | | | GACTGGGCTGACC | |
| B | B3 | 5′I2E3B-U3 | Intron 2> | GTAAAACGACGGCCAGTGGGG | 17 |
| | | | | GACGGTGCTGTCC | |
| B | B3 | 5′I2E3B-U4 | Intron 2> | GTAAAACGACGGCCAGTGGGG | 18 |
| | | | | GACGGTGCTGACC | |
| B | B1SA | B1SA-U1 | Intron 1 > | GTAAAACGACGGCCAGTGGGA | 19 |
| | | | | GGGAAATGGCCTCT | |
| B | B1SA | B1SA-U2 | Intron 1> | GTAAAACGACGGCCAGTGGcA | 20 |
| | | | | GGGAAATGGCCTCT | |
| B | B1SA | B1SA-UR3 | <Intron 3 | CAGGAAACAGCTATGACCACC | 21 |
| | | | | ATCCCCGGCGACCTAT | |
| B | B1SA | B1SA-UR4 | <Intron 3 | CAGGAAACAGCTATGACCACC | 22 |
| | | | | ATCCCGGGCGATCTAT | |
| B | B4 | B1e4-86 | Intron 3> | CACTGTGAAGCTCTCCACATG | 23 |
| | | | | GGTGGTCCTAGGGTG | |
| B | B4 | B1e4 | <Intron 4 | GCTCCTGCTTTCCCTGAG | 24 |
| C | C1 | 5′CIN1-61-1 | Intron 1> | CGAGGTGCCCGCCCGGCGA | 25 |
| C | C1 | 5′CIN1-61-2 | Intron 1> | CGAGGGGCCCGCCCGGCGA | 26 |
| C | C1 | 3′BCIN3-12 | <Intron 3 | AGATGGGGAAGGCTCCCCACT | 27 |
| C | C2 | 5′I1E2C-U1 | Intron 1> | GTAAAACGACGGCCAGTTCGG | 28 |
| | | | | GCGGGTCTCAGCC | |
| C | C2 | 3′I2E2ABC-1UR | <Intron 2 | CAGGAAACAGCTATGACCAGT | 29 |
| | | | | CGTGACCTGCGCCCC | |
| C | C2 | 3′I2E2ABC-2UR | <Intron 2 | CAGGAAACAGCTATGACCAGT | 30 |
| | | | | CCTGACCTGCGCCCC | |
| C | C3 | 5′I2E3C-U1 | Intron 2> | GTAAAACGACGGCCAGTTGAC | 31 |
| | | | | CACGGGGGCGGG | |
| C | C3 | 5′I2E3C-U2 | Intron 2> | GTAAAACGACGGCCAGTTGAC | 32 |
| | | | | CGCGGGGGCGGG | |
| C | C3 | 3′bcI3-UR | <Intron 3 | CAGGAAACAGCTATGACCAAG | 33 |
| | | | | ATGGGGAAGGCTCCCCACT | |
| DQA1 | DQA2 | 5DQA2 | Intron 1> | GTAAAACGACGGCCAGTCTGT | 34 |
| | | | | TCTCTGCCTTCCTGC | |
| DQA1 | DQA2 | 3DQA2 | <Intron 2 | GAT CTG GGG ACC TCT | 35 |
| | | | | TGG | |
| DQA1 | DQA23 | DQA23U1 | Intron 1> | GTAAAACGACGGCCAGTCCTG | 36 |
| | | | | TTCTCCGCCTACCTG | |
| DQA1 | DQA23 | DQA23U2 | Intron 1> | GTAAAACGACGGCCAGTCCTG | 37 |
| | | | | TTCTCCGCCTTCCTG | |
| DQA1 | DQA23 | DQA23U3 | Intron 1> | GTAAAACGACGGCCAGTCCTG | 38 |
| | | | | TTCTCTGCCTTCCTGC | |
| DQA1 | DQA23 | DQA23U4 | Intron 1> | GTAAAACGACGGCCAGTCCTG | 39 |
| | | | | TTCTCCACCTTCTTGC | |
| DQA1 | DQA23 | DQA23U5 | Intron 1> | GTAAAACGACGGCCAGTCCTG | 40 |
| | | | | TTCTCCACCTTCCTGC | |
| DQA1 | DQA23 | DQA23UR6 | <Exon 3 | CAGGAAACAGCTATGACCACC | 41 |
| | | | | CAGTGTTTCAGAAGAGGCT | |
| DQB1 | DQB1 | DQB1-U1 | Intron 1> | GTAAAACGACGGCCAGTCTGA | 42 |
| | | | | CTGGCCCGTGATTCC | |
| DQB1 | DQB1 | DQB1-U2 | Intron 1> | GTAAAACGACGGCCAGTCTGA | 43 |
| | | | | CCGGCCGGTGATTCC | |
| DQB1 | DQB1 | DQB1-U3 | Intron 1> | GTAAAACGACGGCCAGTCTGA | 44 |
| | | | | CTGGCCGGTGATTCC | |
| DQB1 | DQB1 | DQB1-4 | <3′ Exon 2 | CAGGATCCCGCGGTACGCCA | 45 |
| DQB1 | DQB1 | DQB1-5 | <3′ Exon 2 | GTCGTGCGGAGCTCCAACTG | 46 |
| Univ SEQ | 86 | 86 | DR Codon 86 | CACTGTGAAGCTCTCCA | 47 |

| | | | | |
|---|---|---|---|---|
| Univ SEQ | M13F | M13 Universal | GTAAAACGACGGCCAGT | 48 |
| Univ SEQ | M13Ra | M13R Universal | CAGGAAACAGCTATGACCA | 49 |

| | | | | | |
|---|---|---|---|---|---|
| A | A4b | A1e4-1-b | Intron 3> | GCTGGTGGAGTGTCCCATG | 50 |
| A | A4b | A1e4-2-b | Intron 3> | GCTGGAGGAGTGTCCCATG | 51 |
| A | A4b | A1e4-3 | <Intron 4 | GGTCTCCAGAGAGGCTCCTG | 52 |
| A | A4b | A1e4-4 | <Intron 4 | AAGGTCAGAGAGGCTCCTGC | 53 |
| B | B1SAb | B1SA-1 | Intron 1> | GGGAGGGAAATGGCCTCT | 54 |
| B | B1SAb | B1SA-2 | Intron 1> | GGcAGGGAAATGGCCTCT | 55 |
| B | B1SAb | B1SA-R3 | <Intron 3 | CCATCCCCGGCGACCTAT | 56 |
| B | B1SAb | B1SA-R4 | <Intron 3 | CCATCCCGGGCGATCTAT | 57 |
| B | B4b | B1e4-B | Intron 3> | CATGGGTGGTCCTAGGGTG | 58 |
| B | B4b | B1e4 | <Intron 4 | GCTCCTGCTTTCCCTGAG | 59 |
| DQB1 | DQB1b | DQB1-1b | Intron 1> | CTGACTGGCCCGTGATTCC | 60 |
| DQB1 | DQB1b | DQB1-2b | Intron 1> | CTGACCGGCCGGTGATTCC | 61 |
| DQB1 | DQB1b | DQB1-3b | Intron 1> | CTGACTGGCCGGTGATTCC | 62 |
| DQB1 | DQB1b | DQB1-4 | <3′ Exon 2 | CAGGATCCCGCGGTACGCCA | 63 |
| DQB1 | DQB1b | DQB1-5 | <3′ Exon 2 | GTCGTGCGGAGCTCCAACTG | 64 |
| DQA1 | DQA23b | DQA23-1 | Intron 1> | CCTGTTCTCCGCCTACCTG | 65 |
| DQA1 | DQA23b | DQA23-2 | Intron 1> | CCTGTTCTCCGCCTTCCTG | 66 |
| DQA1 | DQA23b | DQA23-3 | Intron 1> | CCTGTTCTCTGCCTTCCTGC | 67 |
| DQA1 | DQA23b | DQA23-4 | Intron 1> | CCTGTTCTCCACCTTCTTGC | 68 |
| DQA1 | DQA23b | DQA23-5 | Intron 1> | CCTGTTCTCCACCTTCCTGC | 69 |
| DQA1 | DQA23b | DQA23R6 | <Exon 3 | CCCAGTGTTTCAGAAGAGGCT | 70 |

### EXAMPLE 1

Example 1 demonstrates bi-directional universal sequencing of exon 2 of a class I HLA-B locus, utilizing the PCR amplification strategy as shown in FIG. 2, wherein the two primers utilized in the PCR amplification contained M13F and M13Ra universal sequencing tags, respectively.

FIG. 10 depicts an agarose gel confirming independent amplification of exons 2 and 3 of 3 samples. Exons 2 and 3 were amplified together in a primary PCR utilizing the B1 primer set (see Table 1), and this amplicon was used as a template for a secondary PCR utilizing the B2 or B3 primer sets (Table 1), for amplification of exon 2 or exon 3, respectively, wherein these secondary amplicons were provided with M13F and M13Ra universal sequencing tags on opposing ends thereof. The relative size of the secondary amplicons (rows 2 and 3 of the gel) were consistent with the control bands (row 1 of the gel). FIG. 11 demonstrates that the PCR product of exon 2 was of sufficient quality for DNA sequencing as described herein, utilizing M13F and M13Ra sequencing primers. The PCR product of exon 3 was also of sufficient quality for DNA sequencing (not shown). From this data, the HLA type of B*4201, B*5801 was determined by the software, as evidenced by the allele combination perfectly matching these two known alleles.

### EXAMPLE 2

Example 2 demonstrates bi-directional universal sequencing of exon 3 of a class I HLA-Cw locus, utilizing the PCR amplification strategy of FIG. 2, wherein the two primers utilized in the PCR amplification contained M13F and M13Ra universal sequencing tags, respectively.

FIG. 12 depicts an agarose gel confirming independent amplification of exons 2 and 3 of 5 samples. Exons 2 and 3 were amplified together in a primary PCR utilizing the C1 primer set (see Table 1), and this amplicon was used as a template for a secondary PCR utilizing the C2 or C3 primer sets (Table 1), for amplification of exon 2 or exon 3, respectively, wherein these secondary amplicons were provided with M13F and M13Ra universal sequencing tags on opposing ends thereof. The relative size of the secondary amplicons (rows 2 and 3 of the gel) were consistent with the control bands (row 1 of the gel). FIG. 13 demonstrates that the PCR product of exon 3 was of sufficient quality for DNA sequencing as described herein, utilizing, sequencing primers M13F (top chromatogram) and M13Ra (bottom chromatogram). The PCR product of exon 2 was also of sufficient quality for DNA sequencing (not shown). From this data, the HLA type of C*0401, C*0602 was determined by the software, as evidenced by the allele combination perfectly matching these two known alleles.

Taken together, Examples 1 and 2 demonstrate the ability to obtain an HLA class I type utilizing sequence-based typing by bi-directionally universally sequencing any exon of an HLA class I locus utilizing the methods of the present invention.

### EXAMPLE 3

Example 3 demonstrates universal sequencing of a single amplicon comprising at least two exons of a class I HLA locus, utilizing the PCR amplification strategy of as shown in FIG. 3, wherein the two opposing primers utilized in the PCR amplification contained M13F and M13Ra universal sequencing tags, respectively, and the amplicon so produced contained exons 2 and 3 of HLA-B locus.

FIG. 14 depicts an agarose gel confirming independent amplification of exons 2 and 3 of 12 samples. Exons 2 and 3 were amplified by the strategy of FIG. 3 using B1SA primer set (Table I), and the amplicon was provided with M13F and M13Ra universal sequencing tags on opposing ends thereof. The relative size of the amplicon so produced from each sample (rows 5-7 of the gel) were consistent with the control bands (row 1 of the gel). FIG. 15 demonstrates that the PCR product of exons 2 and 3 was of sufficient quality for DNA sequencing as described herein, utilizing M13F and M13Ra sequencing primers. The top chromatogram of exon 2 was sequenced using the M13F tag on the 5′ end of the amplicon, whereas the bottom chromatogram of exon 3 was the same amplicon sequenced using the M13Ra tag of the 3′ end of the amplicon. From this data, the HLA type of B*0801, B*3501 was determined by the software, as evidenced by the allele combination perfectly matching these two known alleles.

Thus, Example 3 clearly demonstrates that the present invention is not limited to amplification of a single exon; rather, the present invention includes methods of HLA typing utilizing an amplicon comprising multiple HLA exons.

### EXAMPLE 4

Example 4 demonstrates production of two amplicons of class I HLA-B locus in a single PCR reaction, followed by universal sequencing of a first amplicon comprising exons 2 and 3 of HLA-B using opposing universal primers and uni-directional universal sequencing of a second amplicon, comprising exon 4 of HLA-B. Example 4 utilizes the PCR amplification strategy as shown in FIG. 4. The two primers utilized for amplification of the first amplicon (comprising exons 2 and 3) contained M13 forward and reverse as universal sequencing tags, respectively, while one of the primers utilized for amplification of the second amplicon (comprising exon 4) contained the 86 primer as a universal sequencing tag.

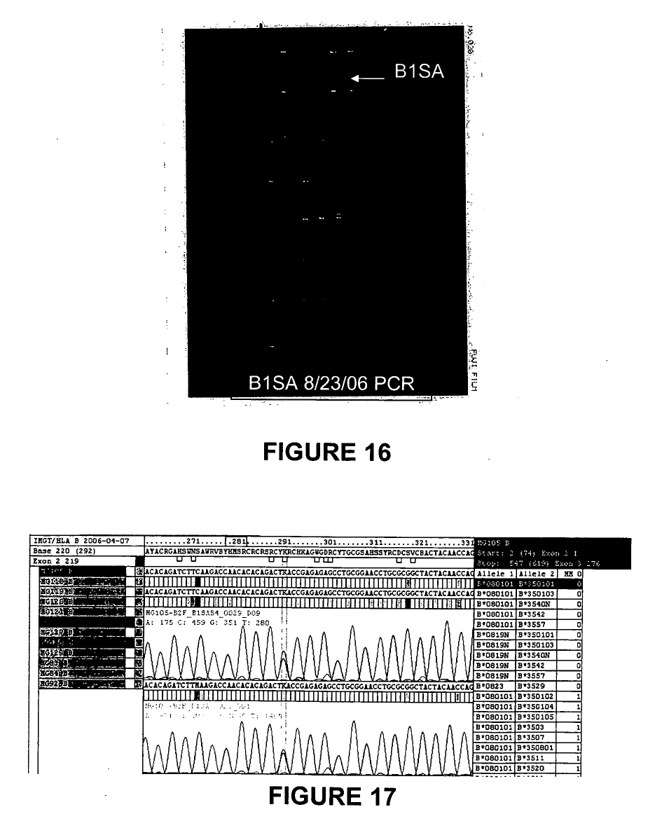

FIG. 16 depicts an agarose gel confirming independent amplification of two amplicons, one comprising exons 2 and 3 and the other comprising exon 4, of 12 samples. The two amplicons were amplified by the strategy of FIG. 4 using B1SA and B4 primer sets (Table I), and the first amplicon was provided with M13F and M13Ra universal sequencing tags on opposing ends thereof, whereas the second amplicon was provided with the 86 primer tag on one end thereof. The relative size of the amplicons so produced from each sample (rows 5-6 of the gel) were consistent with the control bands (row 1 of the gel), and the double bands were indicative of the two amplicons tagged with different universal sequencing primers. FIG. 17 demonstrates that the PCR products were of sufficient quality for DNA sequencing as described herein, utilizing M13F, M13Ra and 86 sequencing primers. The top chromatogram illustrates the sequencing of both amplicons, whereas the bottom chromatogram illustrates the sequencing of a control reaction, in which only the first amplicon was produced (i.e., in a similar manner to that shown in FIG. 3). From this data, the HLA type of B*0801, B*3501 was determined by the software, as evidenced by the allele combination perfectly matching these two known alleles.

This example demonstrates the ability to utilize any sequence that is not complementary to the DNA template of the locus being amplified as a universal sequencing tag, as primer 86 was utilized as a universal sequencing tag in amplification and sequencing of the second amplicon.

The amplification strategy of FIG. 4 has also been used to amplify more than one amplicon containing exons of HLA-A as well as HLA-B, and such amplicons were also of sufficient quality for DNA sequencing as described herein (data not shown).

### EXAMPLE 5

Example 5 demonstrates unidirectional universal sequencing of exon 4 of a class I HLA-A locus, utilizing the PCR amplification strategy as shown in FIG. 5, wherein one the two primers utilized in the PCR amplification contained the 86 primer universal sequencing tag.

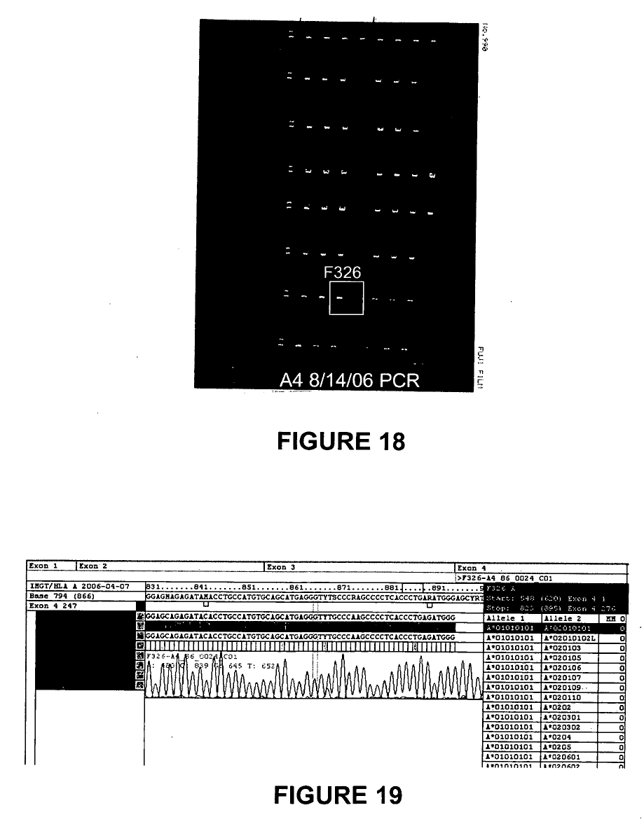

FIG. 18 depicts an agarose gel confirming independent amplification of exon 4 of 25 samples. Exon 4 was amplified by the strategy of FIG. 5 using A4 primer set (Table I), and the amplicon was provided with an 86 primer tag on one end thereof. The relative size of the amplicon so produced from each sample (rows 2-5 of the gel) were consistent with the control bands (row 1 of the gel). FIG. 19 demonstrates that the PCR product of sample F326 (labeled on FIG. 18) was of sufficient quality for DNA sequencing as described herein, utilizing 86 sequencing primer. From this data, the HLA type of A*0101, A*0201 was determined by the software, as evidenced by the allele combination perfectly matching these two known alleles.

Again, this example further demonstrates that any locus specific primer sequence can be utilized as a universal sequencing primer for any locus other than its endogenous locus.

### EXAMPLE 6

Example 6 demonstrates uni-directional universal sequencing of exon 4 of a class I HLA-B locus, utilizing the PCR amplification strategy as shown in FIG. 5, wherein one the two primers utilized in the PCR amplification contained the 86 primer universal sequencing tag.

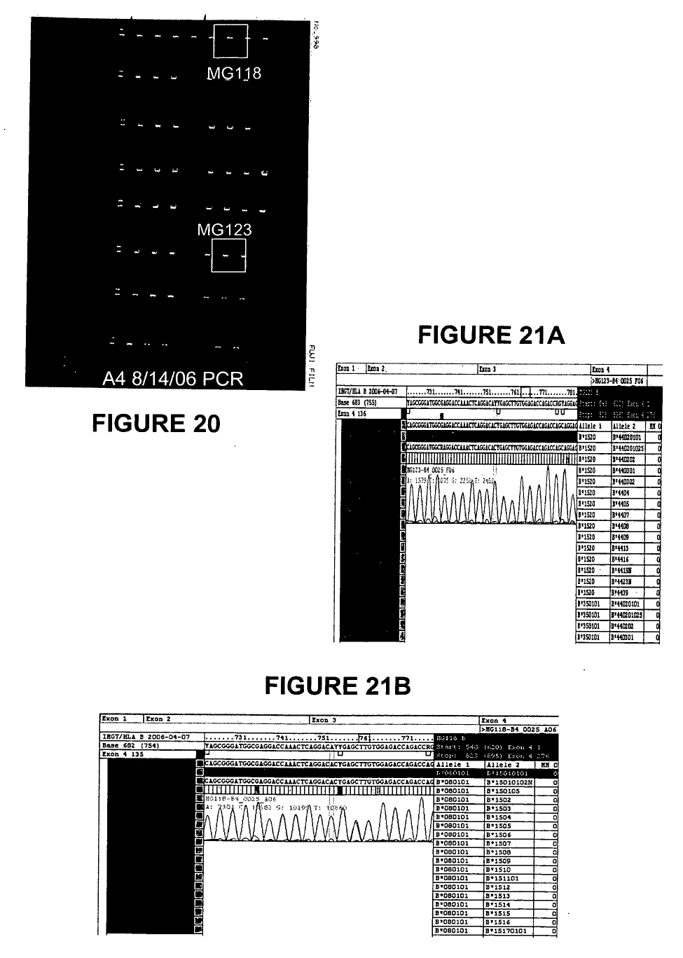

FIG. 20 depicts an agarose gel confirming independent amplification of exon 4 of 25 samples. Exon 4 was amplified by the strategy of FIG. 5 using B4 primer set (Table I), and the amplicon was provided with 86 sequencing tag on one end thereof. The relative size of the amplicon so produced from each sample (rows 6-8 of the gel) were consistent with the control bands (row 1 of the gel). FIG. 21 demonstrates that the PCR products of exon 4 for two of the samples (MG123 in FIG. 21A and MG118 in FIG. 21B; see row 6 of the gel in FIG. 20) were of sufficient quality for DNA sequencing as described herein, utilizing 86 sequencing primers. From this data, the HLA type of B*3501, B*4402 for FIGS. 21A and B*0801, B*1501 were determined by the software, as evidenced by the allele combination perfectly matching these two known alleles.

Again, this example further demonstrates the ability to utilize any sequence that is not complementary to the DNA template of the locus being amplified as a universal sequencing tag.

### EXAMPLE 7

Example 7 demonstrates uni-directional universal sequencing of exon 2 of a class II HLA-DQA1 locus, utilizing the PCR amplification strategy as shown in FIG. 6, wherein one the two primers utilized in the PCR amplification contained the M13F universal sequencing tag.

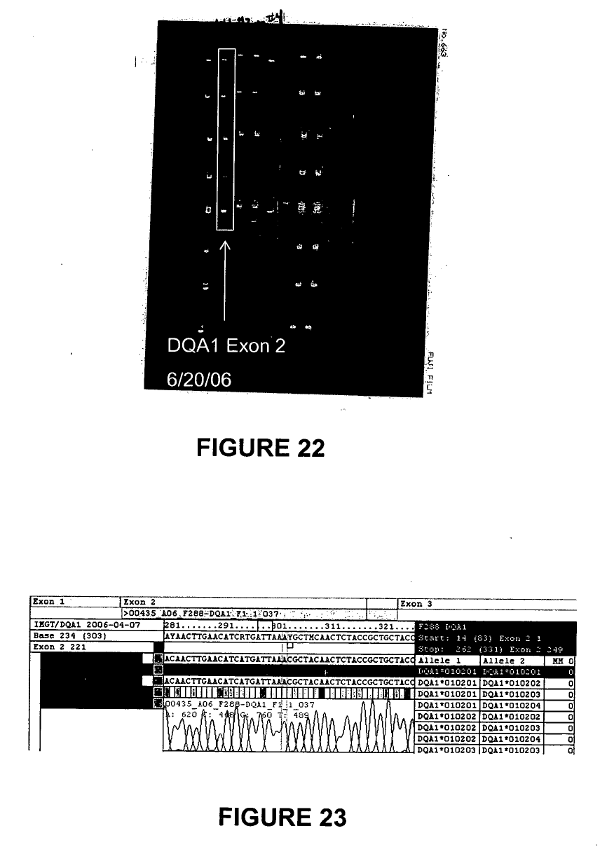

FIG. 22 depicts an agarose gel confirming independent amplification of exon 2 of 5 samples. Exon 2 was amplified by the strategy of FIG. 6 using DQA2 primer set (Table I), and the amplicon was provided with an 86 primer tag on one end thereof. The relative size of the amplicon so produced from each sample (row 2 of the gel) were consistent with the control bands (row 1 of the gel). FIG. 23 demonstrates that the PCR product was of sufficient quality for DNA sequencing as described herein, utilizing 86 sequencing primer. From this data, the HLA type of DQA*0102, DQA*0102 was determined by the software, as evidenced by the allele combination perfectly matching these two known alleles.

Example 7 demonstrates that universal sequencing primers can be utilized in the methods of typing HLA class II of the present invention.

### EXAMPLE 8

Example 8 demonstrates uni-directional universal sequencing of exon 2 of a class II HLA-DQB1 locus, utilizing the PCR amplification strategy as shown in FIG. 6, wherein one of the two primers utilized in the PCR amplification contained the M13F universal sequencing tag.

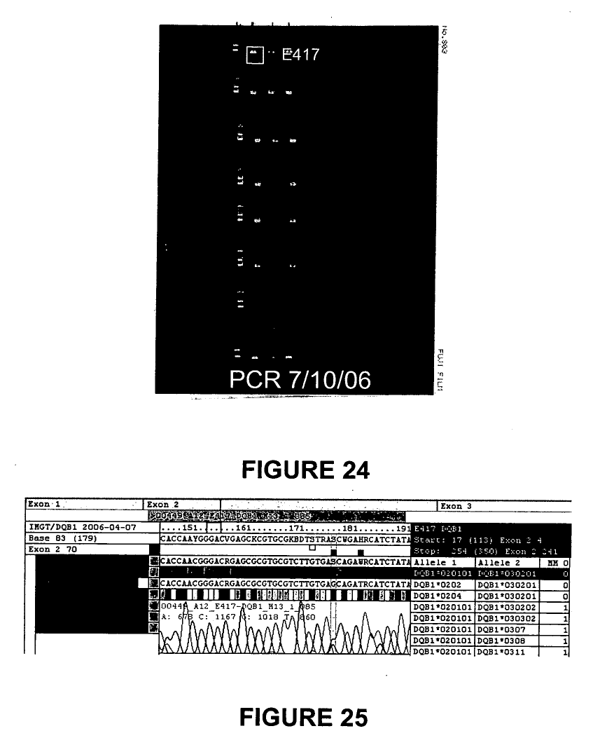

FIG. 24 depicts an agarose gel confirming independent amplification of exon 2 of 8 samples. Exon 2 was amplified by the strategy of FIG. 6 using DQB1 primer set (Table I), and the amplicon was provided with an M13F primer tag on one end thereof. The relative size of the amplicon so produced from each sample (rows 2-4 of the gel) were consistent with the control bands (row 1 of the gel). FIG. 25 demonstrates that the PCR product was of sufficient quality for DNA sequencing as described herein, utilizing M13F sequencing primer. From this data, the HLA type of DQB*0201, DQB*0302 was determined by the software, as evidenced by the allele combination perfectly matching these two known alleles.

### EXAMPLE 9

Example 9 demonstrates universal sequencing of a single amplicon comprising at least two exons of a HLA class II locus using opposing universal primers, utilizing the PCR amplification strategy as shown in FIG. 8, wherein the two primers utilized in the PCR amplification contained M13 forward and reverse universal sequencing tags, respectively, and the amplicon so produced contained exons 2 and 3 of HLA-DQA1 locus.

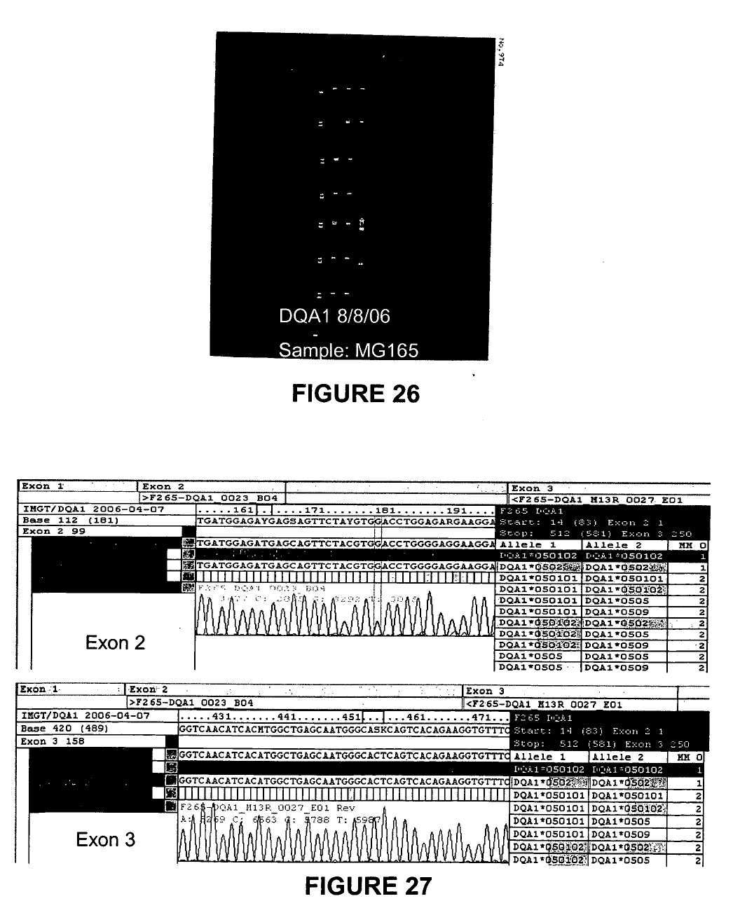

FIG. 26 depicts an agarose gel confirming independent amplification of exons 2 and 3 of 18 samples. Exons 2 and 3 were amplified by the strategy of FIG. 8 using DQA23 primer set (Table I), and the amplicon was provided with M13F and M13Ra universal sequencing tags on opposing ends thereof. The relative size of the amplicon so produced from each sample (rows 2-4 of the gel) were consistent with the control bands (row 1 of the gel). FIG. 27 demonstrates that the PCR product of exons 2 and 3 was of sufficient quality for DNA sequencing as described herein, utilizing M13F and M13Ra sequencing primers. The top chromatogram of exon 2 was sequenced using the M13F tag on the 5′ end of the amplicon, whereas the bottom chromatogram of exon 3 was the same amplicon sequenced using the M13Ra tag of the 3′ end of the amplicon. From this data, the HLA type of DQA1*0501, DQA1*0501 was determined by the software, as evidenced by the allele combination perfectly matching these two known alleles.

Thus, it should be apparent that there has been provided in accordance with the present invention a method of typing a sample for its HLA class I or class II type which satisfies the objectives and advantages set forth above. Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

1. A method for typing HLA class I or class II alleles, the method comprising the steps of:
providing a first primer for locus specific PCR amplification of at least one exon of an HLA locus, wherein the first primer comprises:
a first portion complementary to the HLA DNA template comprising an HLA locus, wherein the first portion is located at a 3′ end terminus of the primer; and
a second portion not complementary to the HLA DNA template comprising an HLA locus, wherein the second portion comprises a forward generic sequencing primer sequence, and wherein the second portion is located at a 5′ end terminus of the primer;
providing a second primer for locus specific PCR amplification of at least one exon of an HLA locus, wherein the second primer comprises:
a first portion complementary to the HLA DNA template comprising an HLA locus, wherein the first portion is located at a 3′ end terminus of the primer; and
a second portion not complementary to the HLA DNA template comprising an HLA locus, wherein the second portion comprises a reverse generic sequencing primer sequence different from the second portion of the first primer, and wherein the second portion is located at a 5′ end terminus of the primer;
providing a sample comprising at least one HLA allele;
amplifying at least one exon of the at least one HLA allele in a locus specific fashion utilizing the two primers, thereby providing an amplicon having two generic sequencing primer sites at the opposing terminus of the amplicon such that the generic sequencing primer sites flank at least one exon to be sequenced; and
DNA sequencing the amplicon utilizing generic sequencing primers corresponding to the second portions of the first and second primers to obtain the sequence of at least one exon of the HLA locus.

2. The method of claim 1, wherein the method further comprises the step of analyzing the DNA sequence of the amplicon so as to provide an HLA class type for the amplicon.

3. The method of claim 1, wherein the HLA allele is selected from the group consisting of HLA-A, HLA-B, HLA-Cw, HLA-DQA1, HLA-DRB1, HLA-DRB3, HLA-DRB4, HLA-DRB5, and HLA-DQB1.

4. The method of claim 1, wherein at least exon 2 of the at least one HLA allele is amplified, and wherein the first portion of the first primer is complementary to a portion of intron 1, and the first portion of the second primer is complementary to a portion of intron 2.

5. The method of claim 1, wherein at least exons 2 and 3 of the at least one HLA allele are amplified.

6. The method of claim 5, wherein the first portion of the first primer is complementary to a portion of intron 1, and the first portion of the second primer is complementary to a portion of intron 3.

7. The method of claim 1, wherein at least exon 4 of the at least one HLA allele is amplified.

8. The method of claim 1, wherein the first portion of at least one of the two primers comprises a nucleotide sequence of at least one of SEQ ID NOS:3-11, 24-27, 35, 45, 46, and 50-70.

9. The method of claim 1, wherein the second portion of at least one of the two primers comprises a nucleotide sequence of at least one of SEQ ID NOS:47-49.

10. The method of claim 1, wherein at least one of the first and second primers comprises a nucleotide sequence of at least one of SEQ ID NOS:1, 2, 12-23, 28-23 and 36-44.

11. The method of claim 1, wherein the step of amplifying at least one exon of the at least one HLA allele is performed in a single PCR reaction step.

12. The method of claim 1, further comprising the steps of:
providing a third primer for locus specific PCR amplification of at least one exon of the HLA locus other than the at least one exon in the first amplicon, wherein the third primer comprises:
a first portion complementary to the HLA DNA template comprising an HLA locus, wherein the first portion is located at a 3′ end terminus of the third primer; and
a second portion not complementary to the HLA DNA template comprising an HLA locus, wherein the second portion comprises a forward generic sequencing primer sequence different from the second portions of the first and second primers, and wherein the second portion is located at a 5′ end terminus of the third primer;
providing a fourth primer for locus specific PCR amplification of at least one exon other than exon 2 of an HLA locus, wherein the fourth primer comprises:
a first portion complementary to the HLA DNA template comprising an HLA locus, wherein the first portion is located at a 3′ end terminus of the fourth primer; and
a second portion not complementary to the HLA DNA template comprising an HLA locus, wherein the second portion comprises a reverse generic sequencing primer sequence different from the second portions of the first, second and third primers, and wherein the second portion is located at a 5′ end terminus of the fourth primer;
amplifying the at least one exon of the at least one HLA allele in a locus specific fashion utilizing the third and fourth primers, thereby providing a second amplicon having two generic sequencing primer sites at the opposing terminus of the amplicon, wherein the first and second amplicons are amplified in a single amplification reaction; and
DNA sequencing the second amplicon utilizing generic sequencing primers corresponding to the second portions of the third and fourth primers.

13. The method of claim 12, wherein the first portion of at least one of the third and fourth primers comprises a nucleotide sequence of at least one of SEQ ID NOS:3-11, 24-27, 35, 45, 46, and 50-70.

14. The method of claim 12, wherein the second portion of at least one of the third and fourth primers comprises a nucleotide sequence of at least one of SEQ ID NOS:47-49.

15. The method of claim 12, wherein at least one of the third and fourth primers comprises a nucleotide sequence of at least one of SEQ ID NOS:1, 2, 12-23, 28-23 and 36-44.

16. The method of claim 1, wherein the HLA allele is a class I allele.

17. The method of claim 1, wherein the HLA allele is a class II allele.

18. A method for determining tissue compatibility, comprising the steps of:
providing a tissue sample comprising at least one HLA allele;
providing a first primer for locus specific PCR amplification of at least one exon of an HLA locus, wherein the first primer comprises:
a first portion complementary to the HLA DNA template comprising an HLA locus, wherein the first portion is located at a 3′ end terminus of the first primer; and
a second portion not complementary to the HLA DNA template comprising an HLA locus, wherein the second portion comprises a forward generic sequencing primer sequence, and wherein the second portion is located at a 5′ end terminus of the first primer;
providing a second primer for locus specific PCR amplification of at least one exon of an HLA locus, wherein the first primer comprises:
a first portion complementary to the HLA DNA template comprising an HLA locus, wherein the first portion is located at a 3′ end terminus of the second primer; and
a second portion not complementary to the HLA DNA template comprising an HLA locus, wherein the second portion comprises a reverse generic sequencing primer sequence, and wherein the second portion is located at a 5′ end terminus of the second primer;
amplifying at least one exon of the at least one HLA allele in a locus specific fashion utilizing the two primers, thereby providing an amplicon having two generic sequencing primer sites at the opposing terminus of the amplicon such that the generic sequencing primer sites flank at least one exon to be sequenced;
DNA sequencing the amplicon utilizing generic sequencing primers to obtain the sequence of at least one exon of the HLA locus; and
comparing the DNA sequence of the amplicon with at least one predetermined tissue sample.

19. The method of claim 18, wherein the HLA allele is selected from the group consisting of HLA-A, HLA-B, HLA-Cw, HLA-DQA1, HLA-DRB1, HLA-DRB3, HLA-DRB4, HLA-DRB5, and HLA-DQB1.

20. The method of claim 18, wherein at least exon 2 of the at least one HLA allele is amplified, and wherein the first portion of the first primer is complementary to a portion of intron 1, and the first portion of the second primer is complementary to a portion of intron 2.

21. The method of claim 18, wherein at least exons 2 and 3 of the at least one HLA allele are amplified.

22. The method of claim 21, wherein the first portion of the first primer is complementary to a portion of intron 1, and the first portion of the second primer is complementary to a portion of intron 3.

23. The method of claim 18, wherein at least exon 4 of the at least one HLA allele is amplified.

24. The method of claim 18, wherein the first portion of at least one of the two primers comprises a nucleotide sequence of at least one of SEQ ID NOS:3-11, 24-27, 35, 45, 46, and 50-70.

25. The method of claim 18, wherein the second portion of at least one of the two primers comprises a nucleotide sequence of at least one of SEQ ID NOS:47-49.

26. The method of claim 18, wherein at least one of the first and second primers comprises a nucleotide sequence of at least one of SEQ ID NOS:1, 2, 12-23, 28-23 and 36-44.

27. The method of claim 18, wherein the step of amplifying at least one exon of the at least one HLA allele is performed in a single PCR reaction step.

28. The method of claim 18, further comprising the steps of:
providing a third primer for locus specific PCR amplification of at least one exon of the HLA locus other than the at least one exon in the first amplicon, wherein the third primer comprises:
a first portion complementary to the HLA DNA template comprising an HLA locus, wherein the first portion is located at a 3′ end terminus of the third primer; and
a second portion not complementary to the HLA DNA template comprising an HLA locus, wherein the second portion comprises a forward generic sequencing primer sequence different from the second portions of the first and second primers, and wherein the second portion is located at a 5′ end terminus of the third primer;
providing a fourth primer for locus specific PCR amplification of at least one exon other than exon 2 of an HLA locus, wherein the fourth primer comprises:
a first portion complementary to the HLA DNA template comprising an HLA locus, wherein the first portion is located at a 3′ end terminus of the fourth primer; and
a second portion not complementary to the HLA DNA template comprising an HLA locus, wherein the second portion comprises a reverse generic sequencing primer sequence different from the second portions of the first, second and third primers, and wherein the second portion is located at a 5′ end terminus of the fourth primer;
amplifying the at least one exon of the at least one HLA allele in a locus specific fashion utilizing the third and fourth primers, thereby providing a second amplicon having two generic sequencing primer sites at the opposing terminus of the amplicon, wherein the first and second amplicons are amplified in a single amplification reaction; and
DNA sequencing the second amplicon utilizing generic sequencing primers corresponding to the second portions of the third and fourth primers.

29. The method of claim 28, wherein the first portion of at least one of the third and fourth primers comprises a nucleotide sequence of at least one of SEQ ID NOS:3-11, 24-27, 35, 45, 46, and 50-70.

30. The method of claim 28, wherein the second portion of at least one of the third and fourth primers comprises a nucleotide sequence of at least one of SEQ ID NOS:47-49.

31. The method of claim 28, wherein at least one of the first and second primers comprises a nucleotide sequence of at least one of SEQ ID NOS:1, 2, 12-23, 28-23 and 36-44.

32. The method of claim 18, wherein the HLA allele is a class I allele.

33. The method of claim 18, wherein the HLA allele is a class II allele.

34. A method for typing HLA class I or class II alleles, the method comprising the steps of:
providing a first primer for locus specific PCR amplification of at least one exon of an HLA class I or class II locus, wherein the first primer comprises:
a first portion complementary to the HLA class I or class II locus DNA template, wherein the first portion is located at a 3′ end terminus of the primer; and
a second portion not complementary to the HLA class I or class II locus DNA template, wherein the second portion comprises a generic sequencing primer sequence, and wherein the second portion is located at a 5′ end terminus of the first primer;
providing a second primer for locus specific PCR amplification of at least one exon of an HLA class I or class II locus, wherein the second primer is complementary to the HLA class I or class II locus DNA template;
providing a sample comprising at least one HLA class I or class II allele;
amplifying at least one exon of the at least one HLA class I or class II allele in a locus specific fashion utilizing the two primers, thereby providing an amplicon having one generic sequencing primer site at one terminus of the amplicon; and
DNA sequencing the amplicon utilizing a locus-specific sequencing primer and a generic sequencing primer corresponding to the second portion of the first primer to obtain the sequence of at least one exon of the HLA class I or class II locus.

35. The method of claim 34, wherein the HLA allele is selected from the group consisting of HLA-A, HLA-B, HLA-Cw, HLA-DQA1, HLA-DRB1, HLA-DRB3, HLA-DRB4, HLA-DRB5, and HLA-DQB1.

36. The method of claim 34, wherein the first portion of the first primer comprises at least one of SEQ ID NOS:3-11, 24-27, 35, 45, 46, and 50-70, and wherein the second primer comprises a nucleotide sequence of at least one of SEQ ID NOS:3-11, 24-27, 35, 45, 46, and 50-70.

37. The method of claim 34, wherein the second portion of the first primer comprises a nucleotide sequence of at least one of SEQ ID NOS:47-49.

38. The method of claim 34, wherein the HLA allele is a class I allele.

39. The method of claim 34, wherein the HLA allele is a class II allele.

40-56. (canceled)


SEQUENCE LISTING (70 sequences)

SEQ ID NO: 1 - DNA, 36 bp, Homo sapiens
cactgtgaagctctccagctggtggagtgtcccatg

SEQ ID NO: 2 - DNA, 36 bp, Homo sapiens
cactgtgaagctctccagctggaggagtgtcccatg

SEQ ID NO: 3 - DNA, 20 bp, Homo sapiens
ggtctccagagaggctcctg

SEQ ID NO: 4 - DNA, 20 bp, Homo sapiens
aaggtcagagaggctcctgc

SEQ ID NO: 5 - DNA, 21 bp, Homo sapiens
agcccatccccgccgacctat

SEQ ID NO: 6 - DNA, 21 bp, Homo sapiens
aggccatccccgccgacctat

SEQ ID NO: 7 - DNA, 21 bp, Homo sapiens
aggccatccccggcgacctat

SEQ ID NO: 8 - DNA, 21 bp, Homo sapiens
aggccatcccgggcgatctat

SEQ ID NO: 9 - DNA, 20 bp, Homo sapiens
gaggagagaggggaccgcag

SEQ ID NO: 10 - DNA, 20 bp, Homo sapiens
gaggagcaaggggaccgcag

SEQ ID NO: 11 - DNA, 20 bp, Homo sapiens
gaggagcgaggggaccgcag

SEQ ID NO: 12 - DNA, 34 bp, Homo sapiens
gtaaaacgacggccagtgcgccgggaggagggtc

SEQ ID NO: 13 - DNA, 36 bp, Homo sapiens
caggaaacagctatgaccagtcgtgacctgcgcccc

SEQ ID NO: 14 - DNA, 36 bp, Homo sapiens
caggaaacagctatgaccagtcctgacctgcgcccc

SEQ ID NO: 15 - DNA, 40 bp, Homo sapiens
caggaaacagctatgaccaagatggggaaggctccccact

SEQ ID NO: 16 - DNA, 34 bp, Homo sapiens
gtaaaacgacggccagtgggggactgggctgacc

SEQ ID NO: 17 - DNA, 34 bp, Homo sapiens
gtaaaacgacggccagtgggggacggtgctgtcc

SEQ ID NO: 18 - DNA, 34 bp, Homo sapiens
gtaaaacgacggccagtgggggacggtgctgacc

SEQ ID NO: 19 - DNA, 35 bp, Homo sapiens
gtaaaacgacggccagtgggagggaaatggcctct

SEQ ID NO: 20 - DNA, 35 bp, Homo sapiens
gtaaaacgacggccagtggcagggaaatggcctct

SEQ ID NO: 21 - DNA, 37 bp, Homo sapiens
caggaaacagctatgaccaccatccccggcgacctat

SEQ ID NO: 22 - DNA, 37 bp, Homo sapiens
caggaaacagctatgaccaccatcccgggcgatctat

SEQ ID NO: 23 - DNA, 36 bp, Homo sapiens
cactgtgaagctctccacatgggtggtcctagggtg

SEQ ID NO: 24 - DNA, 18 bp, Homo sapiens
gctcctgctttccctgag

SEQ ID NO: 25 - DNA, 19 bp, Homo sapiens
cgaggtgcccgcccggcga

SEQ ID NO: 26 - DNA, 19 bp, Homo sapiens
cgaggggcccgcccggcga

SEQ ID NO: 27 - DNA, 21 bp, Homo sapiens
agatggggaaggctccccact

SEQ ID NO: 28 - DNA, 34 bp, Homo sapiens
gtaaaacgacggccagttcgggcgggtctcagcc

SEQ ID NO: 29 - DNA, 36 bp, Homo sapiens; identical to SEQ ID NO: 13

SEQ ID NO: 30 - DNA, 36 bp, Homo sapiens; identical to SEQ ID NO: 14

SEQ ID NO: 31 - DNA, 33 bp, Homo sapiens
gtaaaacgacggccagttgaccacgggggcggg

SEQ ID NO: 32 - DNA, 33 bp, Homo sapiens
gtaaaacgacggccagttgaccgcgggggcggg

SEQ ID NO: 33 - DNA, 40 bp, Homo sapiens; identical to SEQ ID NO: 15

SEQ ID NO: 34 - DNA, 36 bp, Homo sapiens
gtaaaacgacggccagtctgttctctgccttcctgc

SEQ ID NO: 35 - DNA, 18 bp, Homo sapiens
gatctggggacctcttgg

SEQ ID NO: 36 - DNA, 36 bp, Homo sapiens
gtaaaacgacggccagtcctgttctccgcctacctg

SEQ ID NO: 37 - DNA, 36 bp, Homo sapiens
gtaaaacgacggccagtcctgttctccgccttcctg

SEQ ID NO: 38 - DNA, 37 bp, Homo sapiens
gtaaaacgacggccagtcctgttctctgccttcctgc

SEQ ID NO: 39 - DNA, 37 bp, Homo sapiens
gtaaaacgacggccagtcctgttctccaccttcttgc

SEQ ID NO: 40 - DNA, 37 bp, Homo sapiens
gtaaaacgacggccagtcctgttctccaccttcctgc

SEQ ID NO: 41 - DNA, 40 bp, Homo sapiens
caggaaacagctatgaccacccagtgtttcagaagaggct

SEQ ID NO: 42 - DNA, 36 bp, Homo sapiens
gtaaaacgacggccagtctgactggcccgtgattcc

SEQ ID NO: 43 - DNA, 36 bp, Homo sapiens
gtaaaacgacggccagtctgaccggccggtgattcc

SEQ ID NO: 44 - DNA, 36 bp, Homo sapiens
gtaaaacgacggccagtctgactggccggtgattcc

SEQ ID NO: 45 - DNA, 20 bp, Homo sapiens
caggatcccgcggtacgcca

SEQ ID NO: 46 - DNA, 20 bp, Homo sapiens
gtcgtgcggagctccaactg

SEQ ID NO: 47 - DNA, 17 bp, Homo sapiens
cactgtgaagctctcca

SEQ ID NO: 48 - DNA, 17 bp, Homo sapiens
gtaaaacgacggccagt

SEQ ID NO: 49 - DNA, 19 bp, Homo sapiens
caggaaacagctatgacca

SEQ ID NO: 50 - DNA, 19 bp, Homo sapiens
gctggtggagtgtcccatg

SEQ ID NO: 51 - DNA, 19 bp, Homo sapiens
gctggaggagtgtcccatg

SEQ ID NO: 52 - DNA, 20 bp, Homo sapiens
ggtctccagagaggctcctg

SEQ ID NO: 53 - DNA, 20 bp, Homo sapiens
aaggtcagagaggctcctgc

SEQ ID NO: 54 - DNA, 18 bp, Homo sapiens
gggagggaaatggcctct

SEQ ID NO: 55 - DNA, 18 bp, Homo sapiens
ggcagggaaatggcctct

SEQ ID NO: 56 - DNA, 18 bp, Homo sapiens
ccatccccggcgacctat

SEQ ID NO: 57 - DNA, 18 bp, Homo sapiens
ccatcccgggcgatctat

SEQ ID NO: 58 - DNA, 19 bp, Homo sapiens
catgggtggtcctagggtg

SEQ ID NO: 59 - DNA, 18 bp, Homo sapiens
gctcctgctttccctgag

SEQ ID NO: 60 - DNA, 19 bp, Homo sapiens
ctgactggcccgtgattcc

SEQ ID NO: 61 - DNA, 19 bp, Homo sapiens
ctgaccggccggtgattcc

SEQ ID NO: 62 - DNA, 19 bp, Homo sapiens
ctgactggccggtgattcc

SEQ ID NO: 63 - DNA, 20 bp, Homo sapiens
caggatcccgcggtacgcca

SEQ ID NO: 64 - DNA, 20 bp, Homo sapiens
gtcgtgcggagctccaactg

SEQ ID NO: 65 - DNA, 19 bp, Homo sapiens
cctgttctccgcctacctg

SEQ ID NO: 66 - DNA, 19 bp, Homo sapiens
cctgttctccgccttcctg

SEQ ID NO: 67 - DNA, 20 bp, Homo sapiens
cctgttctctgccttcctgc

SEQ ID NO: 68 - DNA, 20 bp, Homo sapiens
cctgttctccaccttcttgc

SEQ ID NO: 69 - DNA, 20 bp, Homo sapiens
cctgttctccaccttcctgc

SEQ ID NO: 70 - DNA, 21 bp, Homo sapiens
cccagtgtttcagaagaggct